(19) United States
(12) Patent Application Publication
(10) Pub. No.: US 2021/0361568 A1
(43) Pub. Date: Nov. 25, 2021

(54) CANNABINOID-CONTAINING PRODUCTS, CONTAINERS, SYSTEMS, AND METHODS

(71) Applicant: HEXO OPERATIONS INC., Ottawa (CA)

(72) Inventors: Max ALSAYAR, Ottawa (CA); François CHOUINARD, Blainville (CA); Justin CONWAY, Carignan (CA); Patrick WOODS, Ottawa (CA); Denis KESERIS, Baie d'Urfe (CA); Stephan GEORGIEV, Saint-Hubert (CA); Bryce DORIN, Ottawa (CA)

(21) Appl. No.: 17/269,644

(22) PCT Filed: Aug. 20, 2019
(86) PCT No.: PCT/CA2019/051128

§ 371 Date: Feb. 19, 2021

Related U.S. Application Data
(60) Provisional application No. 62/719,942, filed on Aug. 20, 2018.

(51) Int. Cl. A61K 9/00; A61K 36/185; A61K 31/352; B65D 51/28; B65D 51/24

(52) U.S. Cl. CPC A61K 9/0095; A61K 36/185; B65D 2215/00; B65D 51/2892; B65D 51/245; A61K 31/352

(57) ABSTRACT

A dosage device that includes a cannabinoid-containing substance enables controlled addition of the cannabinoid-containing substance to a liquid to produce a cannabinoid-containing liquid for ingestion. In an embodiment, such a dosage device is provided with a container that has a container body holding a liquid and a cover to seal the container body. A dosage device may also or instead be provided with a cover that includes a coupling structure to releasably couple the cover to a liquid container and a seal to seal the container. Other embodiments, including methods of use of a container, methods that involve providing a cannabinoid-containing substance and adding the cannabinoid-containing substance to a cover, and product packages that include multiple covers, are also disclosed.

### CROSS-REFERENCE TO RELATED APPLICATION

This application is related to, and claims priority to, U.S. Provisional Patent Application No. 62/719,942, entitled “CANNABINOID-CONTAINING PRODUCTS, CONTAINERS, SYSTEMS, AND METHODS”, and filed on Aug. 20, 2018, the entire contents of which are incorporated by reference herein.

### FIELD

This application relates generally to cannabinoid-containing products, and in particular to liquid formulations for ingestion and related containers, systems, and methods.

### BACKGROUND

The effects of psychoactive compounds such as cannabinoids may be influenced by, among other things, how such compounds are delivered and absorbed. Effects are more time-delayed for orally ingested compounds that are absorbed through the digestive system, for example, compared to inhaled compounds that are absorbed into the blood through the lungs.

Effects are also dependent upon concentration or strength of a mixture such as a drink or other liquid formulation. Although a drink producer might produce drinks of several different strengths, this might only be feasible for a relatively small strength selection in a product line. Producing a product line with more than a certain number of different strengths might not be cost-effective, for example.

### SUMMARY

It may be desirable to support multiple cannabinoid delivery modes or mechanisms for a cannabinoid-containing product. A primary delivery mode could provide a cannabinoid-containing liquid for ingestion, but this could be supplemented with a different delivery mode, such as airborne delivery to provide a cannabinoid-containing aerosol for inhalation. The different delivery modes could deliver the same cannabinoid(s) or different cannabinoids.

According to one aspect of the present disclosure, a product comprises a cannabinoid-containing liquid for ingestion, and an additive for production of a cannabinoid-containing aerosol for inhalation.

Use of such a product for delivery of a cannabinoid-containing liquid for ingestion and a cannabinoid-containing aerosol for inhalation is also contemplated.

A container comprising such a product, as well as use of such a container for delivery of a cannabinoid-containing liquid for ingestion and a cannabinoid-containing aerosol for inhalation, are also disclosed. As an example, a container may include a container body having an interior space with such a product therein, and a cover to seal the container body interior space.

A further aspect of the present disclosure relates to a container comprising: a container body holding therein a cannabinoid-containing liquid for ingestion and an additive for production of a cannabinoid-containing aerosol for inhalation; and a cover releasably coupled to the container body to seal the container body.

A method of use of such a container for delivery of a cannabinoid-containing liquid for ingestion and a cannabinoid-containing aerosol for inhalation could include: opening the container; and inhaling the cannabinoid-containing aerosol; and ingesting the cannabinoid-containing liquid.

A product package could include a number of such containers.

A product package could also include an indicium providing an estimate, or an indicator of an estimate, of effect of either or both of the cannabinoid-containing liquid and the cannabinoid-containing aerosol.

Another aspect relates to a container comprising: a container body holding therein contents comprising a liquid for ingestion; a cover releasably coupled to the container body to seal the container body; and an aerosolizer, packaged with the container, to aerosolize a cannabinoid from the contents for airborne delivery of a cannabinoid-containing aerosol for inhalation.

Another aspect relates to a combination, including: a container; a closure closing the container; a liquid, stored in the container, for ingestion; and a cannabinoid-containing gaseous substance, stored in the container, for inhalation when the closure closing the container is opened. The liquid could be a cannabinoid-containing liquid, in which case the cannabinoid-containing liquid and the cannabinoid-containing gaseous substance could be considered a dual-phase cannabinoid-containing product.

A product according to yet another aspect includes: a container; a liquid for ingestion; and a body of cannabinoid-containing gaseous substance, which is above the liquid in some embodiments, for inhalation as a user drinks the cannabinoid-containing liquid from the container. Such a liquid could be a cannabinoid-containing liquid to provide, in combination with the cannabinoid-containing gaseous substance, a dual-phase cannabinoid-containing product.

The present disclosure also encompasses embodiments that include indicia or indicators of estimated or expected effect. For example, one such embodiment relates to a container that includes a container body holding therein a cannabinoid-containing liquid for ingestion and an additive for production of a cannabinoid-containing aerosol for inhalation, and an indicator of an estimate of effect of either or both of the cannabinoid-containing liquid and the cannabinoid-containing aerosol.

A product package may include a number of such containers, and a package indicator of an estimate of effect of either or both of the cannabinoid-containing liquid and the cannabinoid-containing aerosol.

Another example container includes a container body holding a liquid; a cover releasably coupled to the container body to seal the container body; a dosage device comprising a cannabinoid-containing substance, packaged with the container, to enable controlled addition of the cannabinoid-containing substance to the liquid to produce a cannabinoid-containing liquid for ingestion; and an indicator of any one or more of: an amount of the cannabinoid-containing substance to be released into the container and an estimated cannabinoid concentration in the cannabinoid-containing liquid.

Indicia or indicators may also be applied to other embodiments. For example, another aspect of the present disclosure relates to a product package that includes multiple covers. Each cover includes a coupling structure to releasably couple the cover to a liquid container; a seal to seal the container; and a dosage device comprising a cannabinoid-containing substance to enable controlled addition of the cannabinoid-containing substance to a liquid inside the container to produce a cannabinoid-containing liquid for ingestion. In some embodiments, the covers include covers with dosage devices having one or more of: different cannabinoid-containing substances, different amounts of cannabinoid-containing substances, different estimated effects of cannabinoid-containing substances, and different granularity of control, and the product package further includes an indicator of an estimate of effect of either or both of the cannabinoid-containing liquid and the cannabinoid-containing substance for each cover of the plurality of covers.

Another embodiment relates to a combination that includes a container; a closure closing the container; a liquid, stored in the container, for ingestion; a cannabinoid-containing gaseous substance, stored in the container, for inhalation when the closure closing the container is opened; and an indicator of an estimate of effect of either or both of the cannabinoid-containing liquid and the cannabinoid-containing gaseous substance.

A product according to another aspect includes a container; a liquid for ingestion; a body of cannabinoid-containing gaseous substance for inhalation as a user drinks the liquid from the container; and an indicator of an estimate of effect of the cannabinoid-containing gaseous substance.

A container could also or instead enable a user to mix a cannabinoid-containing liquid. For example, such a container could include: a container body holding a liquid; a cover releasably coupled to the container body to seal the container body; and a dosage device comprising a cannabinoid-containing substance, packaged with the container, to enable controlled addition of the cannabinoid-containing substance to the liquid to produce a cannabinoid-containing liquid for ingestion.

A method of use of a container that has a dosage device, for delivery of a cannabinoid-containing liquid for ingestion, could include operating the dosage device to add a controlled amount of the cannabinoid-containing substance to the liquid to produce the cannabinoid-containing liquid, opening the container in some embodiments, and ingesting the cannabinoid-containing liquid.

A cover for a container could include: a coupling structure to releasably couple the cover to a liquid container; a seal to seal the container; and a dosage device comprising a cannabinoid-containing substance to enable controlled addition of the cannabinoid-containing substance to a liquid inside the container to produce a cannabinoid-containing liquid for ingestion.

A product package could include a number of such covers.

Another aspect of the present disclosure relates to a method that involves providing a cannabinoid-containing substance and adding the cannabinoid-containing substance to such a cover.

Other aspects and features of embodiments of the present disclosure will become apparent to those ordinarily skilled in the art upon review of the following description.

### BRIEF DESCRIPTION OF THE DRAWINGS

Examples of embodiments of the invention will now be described in greater detail with reference to the accompanying drawings.

FIGS. 1A and 1B are top and plan views of an example container according to an embodiment.

FIG. 2 is an exploded isometric view of another example container.

FIGS. 3A to 3C are top, plan, and isometric views of a further example container.

FIG. 4 is an exploded isometric view of another example container.

FIG. 5 is an exploded isometric view of a cover according to another embodiment.

FIG. 6 is an exploded isometric view of another example cover.

FIG. 7 includes top views of cover parts of a further example cover.

FIG. 8 is an exploded isometric view of yet another example cover.

FIG. 9 is an isometric view of another example cover in which cover parts are coupled together by a breakable coupling.

FIG. 10 is a top view of another example container with dosing control.

FIG. 11 is a flow diagram illustrating a method for production of a cannabinoid-containing product.

FIG. 12 is a flow diagram illustrating a method of use according to an embodiment.

FIG. 13 is a flow diagram illustrating a method of use according to another embodiment.

FIG. 14A is an exploded isometric view of another example container.

FIG. 14B is a cross-sectional assembled view of the container in FIG. 14A, taken along the line indicated in FIG. 14A.

FIG. 15 shows a process for producing cannabis products in accordance with a non-limiting embodiment.

FIG. 16 shows a process for spray-drying a cannabinoid emulsion in accordance with a non-limiting embodiment.

FIGS. 17A and 17B show example atomizers that may be used in the process of FIG. 16, in accordance with a non-limiting embodiment.

### DETAILED DESCRIPTION

The present disclosure relates in part to liquid formulations for user consumption by ingestion.

As used herein, the terms “liquid” and “liquid formulation” mean a formulation that flows freely or under pressure but is of a constant volume. A liquid formulation could be a drink, gel, cream, custard, pudding, honey, syrup, broth, soup, gelatin, yogurt, puree, jelly, sauce, liquid eggs, or salad dressing, for example. Although embodiments disclosed refer primarily to liquids for ingestion and may be particularly suited for application to beverages/drinks, other types of liquids are also contemplated.

In some embodiments, the liquid formulation is a drink, such as drinking water, milk (both diary and non-diary), juice, a smoothie, coffee or a caffeinated beverage, tea, herbal tea, a cocoa beverage, a carbonated drink, a nitrogenated drink, an energy drink, a drinkable yogurt, a fermented beverage, or an alcoholic drink. A drink could be a reduced-calorie or zero-calorie drink. An alcoholic drink could include, for example, beer (including lager and/or other types of beer), cider, spirits, wine/fortified wine, and cocktails.

As used herein, terms of degree such as “about”, “approximately” and “substantially” mean a reasonable amount of deviation of the modified term such that the end result is not significantly changed. These terms may refer to a measurable value such as an amount, a temporal duration, and the like, and are meant to encompass variations of +/−0.1% of the given value, preferably +/−0.5%, preferably +/−1%, preferably +/−2%, preferably +/−5% or preferably +/−10%.

For the purpose of this specification, the term “cannabis product(s)” includes goods that are produced from cannabis or hemp, which include plant material, oils, resins, drinks, food additives, edibles, creams, aerosol sprays and vaporization substances, for example. The term “cannabis material(s)” includes cannabis plant material, which refers to plants or parts thereof, and/or materials that are derived from cannabis plant material and are intended for further processing to produce one or more cannabis products.

As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially).

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), cannabinol propyl variant (CBNV), and derivatives thereof. Further examples of cannabinoids are discussed in PCT Patent Application Pub. No. WO2017/190249 and US Patent Application Pub. No. US2014/0271940.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

A liquid formulation provided herein may comprise a cannabinoid in its pure or isolated form or a source material comprising the cannabinoid. Examples of source materials comprising cannabinoids include, but are not limited to: cannabis or hemp plant material such as flowers, seeds, trichomes, and kief; milled cannabis or hemp plant material, extracts obtained from cannabis or hemp plant material, such as resins, waxes and concentrates; and distilled extracts or kief. In some embodiments, pure or isolated cannabinoids and/or source materials comprising cannabinoids may be combined with water, lipids, hydrocarbons such as butane, ethanol, acetone, isopropanol, or mixtures thereof.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some embodiments, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-1-yl)-5-pentyl-1,3-benzenediol); (2) Δ4-cannabidiol (2-(6-isopropenyl-3-methyl-4-cyclohexen-1-yl)-5-pentyl-1,3-benzenediol); (3) Δ3-cannabidiol (2-(6-isopropenyl-3-methyl-3-cyclohexen-1-yl)-5-pentyl-1,3-benzenediol); (4) Δ3,7-cannabidiol (2-(6-isopropenyl-3-methylenecyclohex-1-yl)-5-pentyl-1,3-benzenediol); (5) Δ2-cannabidiol (2-(6-isopropenyl-3-methyl-2-cyclohexen-1-yl)-5-pentyl-1,3-benzenediol); (6) Δ1-cannabidiol (2-(6-isopropenyl-3-methyl-1-cyclohexen-1-yl)-5-pentyl-1,3-benzenediol); and (7) Δ6-cannabidiol (2-(6-isopropenyl-3-methyl-6-cyclohexen-1-yl)-5-pentyl-1,3-benzenediol).

In some embodiments, the cannabinoid is a mixture of tetrahydrocannabinol (THC) and cannabidiol (CBD). The w/w ratio of THC to CBD in the liquid formulation may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1, or about 1000:1. Other w/w ratios are also possible.

In some embodiments, a liquid formulation may include products of cannabinoid metabolism, including 11-hydroxy-Δ9-tetrahydrocannabinol (11-OH-THC).

In some embodiments, a liquid formulation provided herein may comprise one or more encapsulating agents that form a microencapsulation system with the cannabinoid in the liquid formulation. Encapsulating agents may act as a coating material that completely surrounds the cannabinoid, or as a homogeneous or heterogeneous matrix in which the cannabinoid is embedded.

A cannabinoid could be solubilized in a carrier oil or solvent prior to microencapsulation. Carrier oils and solvents suitable for cannabinoids include, but are not limited to, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g. caprylic/capric glycerides), ethanol, acetone, isopropanol, hydrocarbons and any combination thereof.

In some embodiments, the one or more encapsulating agents are film-forming natural or synthetic biopolymers, including proteins, carbohydrates, lipids, fats, and gums, or one or more small-molecule surfactants, or any combination thereof. In some embodiments, the one or more encapsulating agents may be gum arabic; starches such as corn starch; modified starches such as octenyl succinate modified starches; modified cellulose such as methyl cellulose, hydroxypropyl cellulose, methyl hydroxypropyl cellulose, and carboxymethylcellulose; certain types of pectin such as beet pectin; polysaccharides such as maltodextrin and soy soluble polysaccharides; corn fiber gum; globular proteins such as whey protein and whey protein ingredients such as whey protein concentrate, whey protein isolate, and highly purified protein fractions such as β-lactoglobulin and α-lactalbumin; flexible proteins such as gelatin and caseins such as sodium caseinate, calcium caseinate, and purified protein fractions such as β-casein; Tweens (polysorbates) such as Tween 20 (polyoxyethylene sorbitan monolaurate), Tween 40 (polyoxyethylene sorbitan monopalmitate), Tween 60 (polyoxyethylene sorbitan monostearate), Tween 40 (polyoxyethylene sorbitan monopalmitate), Tween 60 (polyoxyethylene sorbitan monostearate), and Tween 80 (polyoxyethylene sorbitan monooleate); sugar esters such as sucrose monopalmitate, sucrose monostearate, sucrose distearate, sucrose polystearate, and sucrose laurate; quillaja saponin (Q-Naturale®) and components thereof; sorbitan esters (Spans) such as Span 20 (sorbitan monolaurate), Span 40 (sorbitan monopalmitate), Span 60 (sorbitan monostearate), Span 80 (sorbitan monooleate); amphiphilic block copolymers; cholesterol; egg yolk- and soy-derived phosphatidylcholines; cyclodextrins such as 2-hydroxypropyl-β-cyclodextrin; lecithin; or any combination thereof.

Microencapsulation techniques may include emulsification and nanoemulsification techniques, mixing, homogenization, injection, spray drying, spray cooling, spray chilling, freeze-drying, air suspension coating, fluidized-bed extrusion, centrifugal extrusion, coacervation, rotational suspension separation, cocrystallization, liposome entrapment, interfacial polymerization, molecular inclusion, microfluidization, ultrasonication, physical adsorption, complex formation, nanosized self-assembly, or any combination thereof. The microencapsulation process may be assisted or accelerated by the application of heat, e.g., through microwave irradiation. Mixing may be modelled using idealized chemical reactors, which may include, but are not limited to, batch reactors, continuous stirred-tank reactors, and plug flow reactors.

In an embodiment, emulsification and spray-drying are used, to provide an emulsification system. Such an emulsification system may be used to add one or more cannabinoid-containing substances to a liquid for ingestion, for example.

Microencapsulation systems may include emulsions, nanoemulsions, micelles, solid lipid nanoparticles, nanostructured lipid carriers, liposomes, nanoliposomes, niosomes, polymer particles, or hydrogel particles.

Cannabinoids are commonly used for recreational purposes to produce physiological effects associated with a feeling of physical and/or emotional satisfaction. Cannabinoids can also be useful in the treatment and/or prophylaxis of a wide variety of diseases or conditions, such as pain, anxiety, inflammation, autoimmune diseases, neurological disorder, psychiatric disorder, malignancy, metabolic disorder, nutritional deficiency, infectious disease, gastrointestinal disorder, or cardiovascular disorder. The cannabinoids may also have application as neuroprotectants, for example, in limiting neurological damage following ischemic insults, such as stroke and trauma, or in the treatment of neurodegenerative diseases such as Alzheimer's disease, Parkinson's disease and HIV dementia.

The present disclosure relates in part to liquid formulations for user consumption by ingestion. A product, for example, could be in the form of a cannabinoid-containing liquid, such as a drink. Although a user or subject could be a human, other types of users or subjects are also contemplated. For example, a user or subject could be an animal such as a companion animal, such as a canine, feline, equine, rodent, reptile, or bird. Use of cannabinoid-containing substances with animals could also or instead extend to agriculture and animals that are grown and harvested for meat production.

In accordance with an aspect of the disclosure, an additional cannabinoid delivery mechanism or mode is provided in connection with a cannabinoid-containing liquid for ingestion. Airborne delivery of a cannabinoid, for example, could provide a user with a faster-acting initial dose of cannabinoid before drinking a beverage. Airborne delivery could also or instead be initiated or stimulated at other times, and not only to deliver an initial dose.

In an embodiment, a cannabinoid-containing liquid, also referred to herein as a liquid formulation, includes an additive for production of a cannabinoid-containing aerosol for inhalation. The cannabinoid-containing liquid and the cannabinoid-containing aerosol could include the same cannabinoid(s) or different cannabinoids. Examples of cannabinoids that could be used in cannabinoid-containing liquids are disclosed elsewhere herein.

A cannabinoid could be dissolved in or otherwise combined with a liquid to form a cannabinoid-containing liquid. A cannabinoid that is soluble in a clear liquid could be dissolved in that liquid to produce a clear cannabinoid-containing liquid, such as a beverage. In other embodiments, a cannabinoid is insoluble, or only partially soluble, in a liquid and is mixed with that liquid. A cannabinoid-containing liquid produced from an insoluble or only partially soluble cannabinoid could be a form of emulsion or suspension, for example.

A cannabinoid-containing liquid could be carbonated or nitrogenated. Carbonation or nitrogenation involves dissolving a carbon-based gas such as carbon dioxide and/or a nitrogen-based gas such as nitrogen gas in the cannabinoid-containing liquid. Increasing pressure of the gas and/or decreasing temperature of the liquid, for example, could increase the amount of gas that is dissolved. A dissolved gas might be useful not only for satisfying user preference for beverage type, but also for producing a cannabinoid-containing aerosol when released from the cannabinoid-containing liquid. When maintained under pressure, in a pressurized container for example, dissolved carbon dioxide gas will normally remain dissolved in the cannabinoid-containing liquid, but at least some of the dissolved carbon dioxide gas will evaporate and “effervesce” at normal atmospheric pressure, when a sealed container is opened and the cannabinoid-containing liquid is de-pressurized. This effervescence or release of gas from the cannabinoid-containing liquid, could potentially be exploited in producing a cannabinoid-containing aerosol.

There are several possible options for such aerosol production from gas that has been dissolved in a liquid. For example, released gas bubbles could propel or carry some cannabinoid-containing liquid from the liquid surface into the air, to thereby aerosolize the cannabinoid-containing liquid. The amount or degree of aerosolization could depend on such characteristics as the size of gas bubbles that break the surface of the cannabinoid-containing liquid, surface tension of the cannabinoid-containing liquid, combined gas and liquid weight of bubbles, and/or possibly others.

Such characteristics could also or instead be taken into account in preparing an additive such as a cannabinoid-containing additive to float on top of the cannabinoid-containing liquid and aid in producing a cannabinoid-containing aerosol when dissolved gas is released from the underlying cannabinoid-containing liquid. A cannabinoid-containing additive could exhibit properties that are more conducive to aerosolization by gas release or effervescence than a cannabinoid-containing liquid that is intended for ingestion. A cannabinoid-containing additive in the form of a film that can coat gas bubbles and/or be propelled into the air as gas bubbles escape the surface of the liquid, for example, could be particularly useful.

In another embodiment, the gas itself includes a cannabinoid. A cannabinoid compound that is stable as a gas, at least long enough to be inhaled by a user, could be mixed with a carbon-based gas or a nitrogen-based gas and dissolved in the cannabinoid-containing liquid. The gas that is released, on de-pressurization in this example, would then include a cannabinoid and produce the cannabinoid-containing aerosol. Some cannabinoids could be soluble in a carbon-based gas such as carbon dioxide, and the same and/or others could be soluble in a nitrogen-based gas. Other gases could also or instead be dissolved in or otherwise mixed with a cannabinoid-containing liquid to provide for airborne delivery of a cannabinoid.

Gas could be released from the cannabinoid-containing liquid when a pressurized container is opened, as described by way of example above. A gas release stimulant could also or instead be added to the cannabinoid-containing liquid to initiate, produce or otherwise stimulate gas release. Those familiar with carbonated beverages, for example, will readily appreciate that adding ice to such beverages stimulates gas release even without a further change in pressure after a container has been opened. Ice is therefore one example of a gas release stimulant that could be used to produce a cannabinoid-containing aerosol from a cannabinoid-containing liquid.

Other gas release stimulants could also or instead be used. For example, a gas release stimulant could be provided as a substance to stimulate the release of gas. Such a substance could be packaged with a cannabinoid-containing liquid. The gas release stimulant could be sealed inside a container with the cannabinoid-containing liquid, in a cover of the container for example, for release into the cannabinoid-containing liquid when the container is to be opened. Examples of how a gas release stimulant substance could be released into the cannabinoid-containing liquid are disclosed elsewhere herein.

Effervescent compounds represent another example of gas release stimulants. One or more effervescent compounds could be used to induce production of carbon-dioxide gas in a cannabinoid-containing liquid and/or a cannabinoid-containing additive. Examples of effervescent compounds include sodium bicarbonate, citric acid, and dry ice. Effervescent compounds could be in the form of tablets, powder, or granules, for example, and could be added to a cannabinoid-containing liquid and/or a cannabinoid-containing additive by a user to induce the production of a cannabinoid-containing aerosol. Thus, gas release as referenced herein encompasses not only release of dissolved gas from a liquid, but also or instead release of gas from another substance such as an effervescent compound.

A gas release stimulant could also or instead be in the form of a device that is packaged with the cannabinoid-containing liquid. Such a device could be sealed inside a container with the cannabinoid-containing liquid for release into the cannabinoid-containing liquid when the container is to be opened. Like a gas release stimulant substance, a gas release stimulant device could be sealed in or otherwise carried by a cover of the container.

A gas release stimulant device could also or instead be activated, by de-pressurization of the container, to stimulate the gas release. Such a device need not necessarily be carried by the cover or otherwise isolated from the cannabinoid-containing liquid, and could instead be in or floating on the liquid and become active only when the container is opened or otherwise de-pressurized.

A gas release stimulant compound or device could serve multiple purposes, and could be infused with or otherwise contain a cannabinoid. A compound or device itself could then be considered a form of additive for producing a cannabinoid-containing aerosol.

In some of these examples, a dissolved gas is an additive to the cannabinoid-containing liquid, and the cannabinoid-containing aerosol is produced by release of the gas from the cannabinoid-containing liquid. In some embodiments, the additive is a cannabinoid-containing additive. The cannabinoid-containing additive could have a lower density or specific gravity than the cannabinoid-containing liquid for example, in which case the additive floats on the cannabinoid-containing liquid.

The cannabinoid-containing liquid and the cannabinoid-containing additive could have different cannabinoid concentrations. A cannabinoid-containing additive that has a higher cannabinoid concentration than the cannabinoid-containing liquid could be preferred for producing the cannabinoid-containing aerosol, for example.

A cannabinoid-containing additive could be or include a cannabinoid concentrate, such as an oil, for example. Other types of concentrates could also or instead be used as a cannabinoid-containing additive.

A gas could be dissolved in either or both of cannabinoid-containing liquid and the cannabinoid-containing additive, and could then produce the cannabinoid-containing aerosol from the cannabinoid-containing additive when released. The gas could, but need not necessarily, include a cannabinoid. The cannabinoid-containing liquid and/or the cannabinoid-containing additive could be carbonated using a carbon-based gas or nitrogenated using a nitrogen-based gas, for example.

As described by way of example above, a gas could be released when a pressurized container is de-pressurized, and/or when a gas release stimulant substance or device is added to the cannabinoid-containing liquid. A gas release stimulant substance or device could also or instead be released into a cannabinoid-containing additive.

Aerosol production is in no way limited to carbonated or nitrogenated liquids. A cannabinoid-containing liquid and/or additive could be suitable for production a cannabinoid-containing aerosol by a nebulizer or other form of device that is capable of converting a liquid or other cannabinoid-containing substance to vapor or aerosol form that can be carried in air. A nebulizer, which could alternatively be referred to as an atomizer, a vaporizer, or an aerosolizer, could be packaged with a cannabinoid-containing product. In some embodiments, such a device is sealed inside a container of the product for release into the product when the container is to be opened. The device could be sealed in or otherwise carried by a cover of the container, for example.

A nebulizer could be activated by de-pressurization of the container. Such a nebulizer could be in or floating on, or otherwise in contact with, the cannabinoid-containing liquid and/or an cannabinoid-containing additive and need not be sealed or otherwise isolated from the cannabinoid-containing liquid or additive.

A nebulizer is an example of a device to generate a cannabinoid-containing aerosol from a liquid, and in other embodiments a substance could also or instead be added to a liquid or additive to generate a cannabinoid-containing aerosol. For example, a cannabinoid-containing liquid and/or additive could be suitable for production a cannabinoid-containing aerosol by addition of a further substance. Such a substance could react with or otherwise interact with a cannabinoid-containing liquid and/or additive to produce the cannabinoid-containing aerosol.

A product of the type disclosed in the examples above, and/or as disclosed by way of example elsewhere herein, could be used for delivery of a cannabinoid-containing liquid for ingestion and a cannabinoid-containing aerosol for inhalation.

Example containers for such products are also disclosed herein, and could be used for delivery of the cannabinoid-containing liquid for ingestion and the cannabinoid-containing aerosol for inhalation.

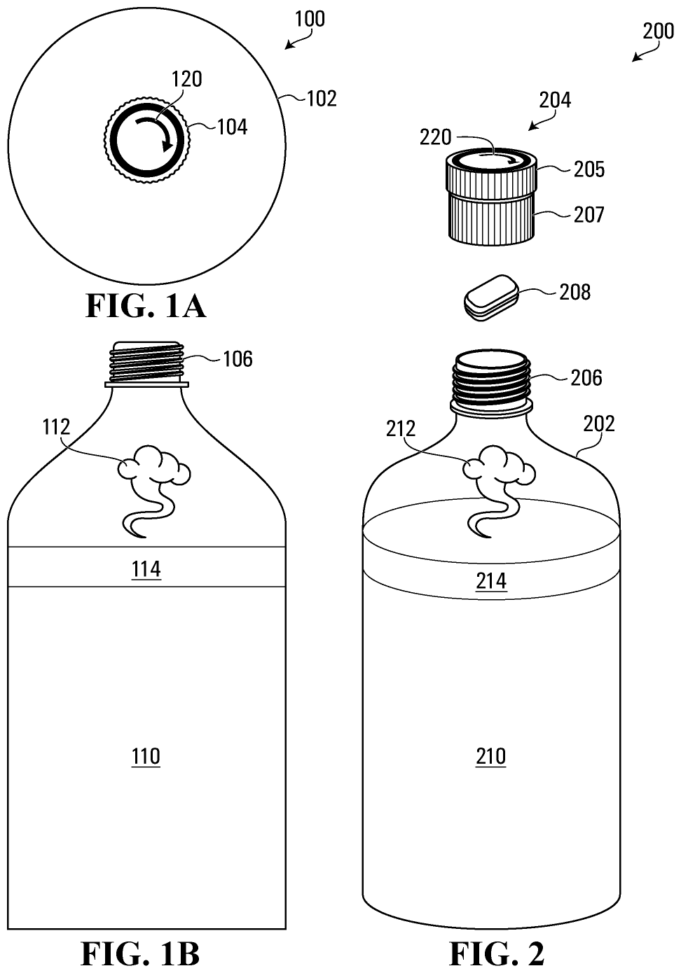

FIGS. 1A and 1B are top and plan views of an example container according to an embodiment. The example container 100 includes a container vessel or body 102 and a cap, stopper, top, or cover 104. The cover 104 is not shown in FIG. 1B so that threads 106 on the neck of the container 102 are visible. These threads 106 cooperate with threads on an inside wall of the cover 104 to releasably attach or couple the cover to the body 102 and seal an inside space of the body. 110 represents a liquid, which is a cannabinoid-containing liquid in some embodiments and is referred to as such herein by way of example, inside the container 100. In other embodiments the container 100 could be filled to a higher or lower level. 112 represents a cannabinoid-containing vapor or aerosol. 114 is an example of an additive, which floats on the cannabinoid-containing liquid 110 in the embodiment shown, but in other embodiments could have a different location relative to the liquid 110, and even be in a different compartment of a container for example.

The container 100 could include a plastic bottle and cover, for example. Bottle bodies and covers are often made from different colors, and/or possibly different types, of plastics. Glass bottles with plastic or metal covers are also common for beverages. Other materials could also or instead be used for a container body 102 and/or a cover 104.

When the container 100 is not in use for consumption of the cannabinoid-containing liquid 110, the cannabinoid-containing liquid remains sealed inside the container, and the container body 102 thus holds therein the cannabinoid-containing liquid for ingestion. In accordance with an aspect of the present disclosure, the container 100 also supports airborne delivery of a cannabinoid-containing aerosol 112, and the container body 102 also holds an additive for production of the cannabinoid-containing aerosol for inhalation. The cannabinoid-containing liquid 110 and the cannabinoid-containing aerosol 112 could include the same cannabinoid(s) or different cannabinoids, as noted above.

In some embodiments, the additive 114 for production of the cannabinoid-containing aerosol is a cannabinoid-containing additive. The cannabinoid-containing liquid 110 and the cannabinoid-containing additive could have the same or different cannabinoid concentrations, for example. A cannabinoid-containing additive that has a higher cannabinoid concentration but a lower density or specific gravity than the cannabinoid-containing liquid 110 and therefore floats on the cannabinoid-containing liquid could be preferred in some embodiments. Such an additive floating on the cannabinoid-containing liquid 110 is shown in FIG. 1B at 114. FIG. 1B is not drawn to scale, and an additive 114 could have a much lower volume relative to the cannabinoid-containing liquid 110 than shown. The additive 114 could form a film on the surface of the cannabinoid-containing liquid 110, for example.

A cannabinoid-containing additive, whether floating on the cannabinoid-containing liquid 110 as shown at 114 or otherwise combined with the cannabinoid-containing liquid, could be or include a cannabinoid concentrate, illustratively an oil. In some embodiments, the cannabinoid-containing additive is aerosolizable by a nebulizer or another type of device for production of the cannabinoid-containing aerosol 112. Such a device could also or instead nebulize or aerosolize the cannabinoid-containing liquid 110 for production of the cannabinoid-containing aerosol 112, in which case the additive is in effect at least somewhat already part of the cannabinoid-containing liquid. In such embodiments, the cannabinoid in the cannabinoid-containing liquid 110 could be considered a form of additive for production of the cannabinoid-containing aerosol 112.

A nebulizer or other form of aerosolization device for producing the cannabinoid-containing aerosol 112 could be packaged with the container 100. Such a device could be packaged inside the container body 102, for example. In an embodiment, a nebulizer is carried by the cover 104. The nebulizer could be releasably carried by the cover 104 for release from the cover when the container 100 is to be opened.

Releasing a nebulizer from the cover 104 could involve, for example, pushing on the cover to force the nebulizer through part of a seal in the cover. The cover could include an internal compartment that remains sealed from the interior space of the container body 102 until a force sufficient to break the seal and release the nebulizer into the container body 102 is applied to the cover.

Nebulizer release could also or instead involve turning the cover 104 in the direction shown by the arrow 120 in FIG. 1A. This could over-tighten the cover 104 to break a seal in the cover, or otherwise cause a nebulizer to be released into the container body 102.

Another possible release mechanism could involve alignment of an internal compartment inside the cover 104 with an aperture in the cover that opens into the container body 102. The nebulizer could be sealed within the compartment until the container 100 is to be opened, and then the cover 104 could be turned in the direction shown by the arrow 120 to align the compartment with the aperture, allowing nebulizer to fall into the container body 102.

The arrow 120 is illustrative of an indicator, icon, label, or indicium of how a nebulizer is to be activated, and could be printed onto the cover 104 or a label that is affixed to the cover, formed in or on the cover, or otherwise placed or mounted in or on the cover. An indicator may be provided elsewhere, such as on a container body 102 and/or on container packaging for example, in other embodiments.

FIG. 2 is an exploded isometric view of another example container 200, which is substantially similar to the container 100 and includes a container body 202 holding a cannabinoid-containing liquid 210 for ingestion, an additive shown by way of example as a cannabinoid-containing additive 214 for producing a cannabinoid-containing aerosol 212, and a neck with threads 206 to engage interior threads on a cover 204. The cover 204 is an example of a multi-part cover, and 208 represents an example of a substance or device that is releasably carried by the cover for release into the container body 202 when the container 200 is to be opened.

Element 208 could be or include a nebulizer. In an embodiment, the lower part 207 of the cover 204 that engages the container body 202 includes a seal to seal the interior space of the container body. The upper part 205 of the cover 204 could provide an interior compartment, outside the seal, to carry the nebulizer. The cover parts 205, 207 could be coupled together to hold the nebulizer in the interior compartment until it is to be released into the container body 202.

For example, the nebulizer could be released from a compartment in the cover 204 by rotating the cover in the direction of the arrow 220. This could over-tighten the cover 204 so that the cover part 207 is moved sufficiently onto the threaded neck of the container body 202 to break a seal in the cover and release the nebulizer. In a multi-part cover 204 as shown, external threads toward or at the top of the cover part 207 could engage with internal threads on the cover part 205 so that the cover part 205 is gradually moved axially toward and over the cover part 207 until a seal in the cover is broken and the nebulizer is released into the container body 202.

A breakable coupling, including a web or more generally one or more connecting segments such as often found at a lower edge of a plastic bottle cover for example, could be useful in reducing the likelihood of inadvertent release of the nebulizer into the container body 202. Release of the nebulizer would then require a force sufficient to break the coupling and turn the cover part 205 relative to the cover part 207.

In another embodiment, an interior shoulder or flange on the cover part 205 could be snapped over an exterior shoulder or flange or one or more protrusions at or near the top of the cover part 207. When the nebulizer is to be released into the container body 202, the cover part 205 could be forced axially toward and over the cover part 207 to break the seal and release the nebulizer into the container body 202. A breakable coupling between the cover parts 205, 207 could be useful in reducing the likelihood of inadvertent axial movement of the cover part 205 relative to the cover part 207 to release the nebulizer into the container body 102.

In such a “push-to-release” embodiment, text or other instructions could be provided on the cover 204 instead of the arrow 220, to indicate that the upper part 205 of the cover is to be pushed downward, toward and over the cover part 205, to release the nebulizer.

Such instructions need not include text, and could be in the form of an icon, drawing, or other indicator that does not require interpretation by a user. A leaf icon or cannabis flower icon next to an arrow pointing axially from a top toward a bottom of a cover, for example, provides an indicator of how a cover that includes a device or substance for release into a container body or liquid, to produce a cannabinoid-containing liquid and/or aerosol for example, is to be activated or used. A leaf or flower icon could similarly be used in conjunction with the arrow 120 (FIG. 1A) and/or the arrow 220 in FIG. 2, in the “turn to release” embodiments shown in those drawings.

An “align to release” mechanism could be provided to release the nebulizer when an internal compartment inside the cover part 205 is aligned with an aperture in the cover part 207 that opens into the container body 202. Instead of the arrow 220 on the top of the cover 204 in the example shown, alignment arrows and/or other indicia could be provided on either or both of the cover parts 205, 207 to indicate proper alignment for release of the nebulizer into the container body 202. An interior shoulder or flange on the cover part 205 could be snapped over an exterior shoulder or flange or one or more protrusions at or near the top of the cover part 207 to couple the cover parts together while still allowing the cover part 205 to be rotated relative to the cover part 207. As in other embodiments, a breakable coupling between the cover parts 205, 207 could be used to reduce the likelihood of inadvertent rotation of the cover part 205 to release the nebulizer. A leaf icon or cannabis flower icon could be provided next to one or more alignment markings as an indicator of how an align to release cover is to be activated or used.

A nebulizer need not necessarily be carried in or by a cover. With reference again to FIGS. 1A and 1B, for example, a nebulizer could be attached to the container 100 or provided in a package that includes one or more containers, and dropped into an open container when the container contents are to be consumed.

It should also be noted that a nebulizer need not necessarily be separated or sealed from the container contents. A nebulizer may be configured to be activated by opening the cover of a container. For example, in some embodiments, the container 100 is pressurized when sealed by the cover 104 and the nebulizer is pressure-sensitive. Such a nebulizer, or potentially another pressure-sensitive device for generating aerosol from container contents, is activated by opening of the container, due to a drop in pressure from opening the container. The nebulizer could then be in or floating on the cannabinoid-containing liquid 110 and/or the cannabinoid-containing additive 114, to produce the cannabinoid-containing aerosol 112 when the container is opened and de-pressurized.

A cannabinoid-containing additive 114, and/or the cannabinoid-containing liquid 110, could be aerosolizable by addition of a further substance, for production of the cannabinoid-containing aerosol 112. Such a substance could be packaged with the container 100, such as inside or attached to the container body 102 or in a package that includes one or more containers.

In an embodiment, an aerosolization substance for producing the cannabinoid-containing aerosol 112, 212 is carried by a cover 104, 204. The substance could be in liquid, tablet, powder, or other form, and be releasably carried by the cover 104, 204 for release from the cover when the container 100, 200 is to be opened. Examples of release mechanisms disclosed herein for nebulizers, cannabinoid doses, and other devices or substances could be applied to releasing an aerosolization substance from a cover. An aerosolization substance that is releasably carried by a cover is another example of element 208 in FIG. 2.

The additive for production of the cannabinoid-containing aerosol 112, 212 could be or include a gas that is dissolved in a liquid. A gas could be dissolved in the cannabinoid-containing additive 114, 214 in which case the cannabinoid-containing aerosol 112. 212 is produced, at least in part, by release of the gas from the cannabinoid-containing additive. A gas could also or instead be dissolved in the cannabinoid-containing liquid 110, 210, and the cannabinoid-containing aerosol 112, 212 is then produced at least in part by release of the gas from the cannabinoid-containing liquid. The same gas or different gases could be dissolved in a cannabinoid-containing additive 114, 214 and the cannabinoid-containing liquid 110, 210.

The gas, or each gas, could itself include a cannabinoid. Examples of a gas include a carbon-based gas and a nitrogen-based gas.

With a container 100, 200 that is pressurized when sealed, de-pressurization could stimulate release of the gas(es), on opening of the container for example.

Gas release could also or instead be stimulated by a gas release stimulant. Ice and effervescent compounds are examples of gas release stimulants that could be added by a user to stimulate gas release. One or more other substances could also or instead be used to stimulate gas release.

A gas release stimulant substance could be packaged with the container 100, 200, such as inside the container body 102, 202 for release into the container body or liquid 110, 210 when the container is to be opened. A gas release stimulant substance could be in liquid, tablet, powder, or other form, and be releasably carried by the cover 104, 204 for release from the cover when the container 100, 200 is to be opened. Such a substance could provide surface irregularities or features that trigger formation of gas bubbles that can subsequently break free and rise to the surface of the cannabinoid-containing liquid 110, 210 or the cannabinoid-containing additive 114, 214. Examples of release mechanisms disclosed herein for nebulizers, cannabinoid doses, and/or other substances could be applied to releasing a gas release stimulant substance from the cover 104, 204. A gas release stimulant substance carried by a cover is a further example of element 208 in FIG. 2.

A device to stimulate the release of a gas is another example of a gas release stimulant. Such a device could be packaged with the container 100, 200, including but not necessarily limited to being packaged inside the container body 102, 202, and could be carried by the cover 104, 204. In some embodiments, the device is releasably carried by the cover 104, 204 for release from the cover when the container is to be opened. Any of the cover release mechanisms disclosed by way of example herein, for a nebulizer for example, could also or instead be applied to a gas release stimulant device.

As noted above for a nebulizer, in an embodiment in which the container 100, 200 is pressurized when sealed by the cover 104, 204, a gas release stimulant device could be pressure-sensitive and activated by opening of the container to stimulate gas release. In this example a drop in pressure from opening a container activates the gas release stimulating device. Such a device may instead be configured in some other way to be activated by opening the cover of a container.

In the example embodiments described above, a container 100, 200 includes a container body 102, 202 holding therein contents that include at least a liquid 110, 210 for ingestion and a cover 104, 204 releasably coupled to the container body to seal the container body. Some embodiments also include an aerosolizer, packaged with the container 100, 200, to aerosolize a cannabinoid from the contents for airborne delivery of a cannabinoid-containing aerosol for inhalation.

The liquid 110, 210 could itself be or include a cannabinoid-containing liquid as described above, from which the aerosolizer is to aerosolize the cannabinoid. In some embodiments, a liquid for ingestion does not contain a cannabinoid, but the contents of the container 100, 200 include a cannabinoid-containing liquid from which the aerosolizer is to aerosolize the cannabinoid. For example, the liquid 110, 210 might not include a cannabinoid, but the liquid 114, 214 includes a cannabinoid for aerosolization in some embodiments.

The cannabinoid-containing liquid could be or include a cannabinoid concentrate, for example.

Examples of an aerosolizer include a nebulizer and a substance to be added to the contents of the container 100, 200 to aerosolize the cannabinoid. Other devices, such as a spray head or pump that is manually operated or otherwise activated or operated, may be used in some embodiments to produce a cannabinoid-containing aerosol from a cannabinoid-containing liquid or additive.

The aerosolizer could also or instead be in the form of a gas that is dissolved in the container contents, in which case the cannabinoid-containing aerosol is produced at least in part by release of the gas from the container contents.

Another form of aerosolizer is a gas release stimulant to stimulate the release of the gas. A gas release stimulant, as disclosed by way of example elsewhere herein, could include any one or more of: a substance to stimulate the release of the gas, a device to stimulate the release of the gas, and a substance to produce a gas.

The aerosolizer could be packaged inside the container body 102, 202, and could be carried by the cover 104, 204 for example. In an embodiment, the aerosolizer is releasably carried by the cover for release from the cover when the container is to be opened. Examples of release mechanisms are disclosed elsewhere herein.

In some embodiments, the aerosolizer is configured to be activated by opening the cover of a container. For a container that is pressurized when sealed by its cover, for example, the aerosolizer could be pressure-sensitive and activated, by opening of the container and an associated drop in pressure, to stimulate the gas release.

Various features relating to containers are described by way of example above. It should be appreciated that the present disclosure is not in any way limited to containers of the type shown in FIGS. 1A, 1B, and 2.

For example, a container need not necessarily include a screw top cover as shown. A cover could instead be press-fit or otherwise coupled to a container body, as in the case of a capped bottle which is opened by prying the bottle cap from the neck or mouth of the bottle. Deformation of the cap could release a device or substance into the bottle. Some embodiments could be implemented in conjunction with stopper-type covers such as corks or clamp-style bottle stoppers.

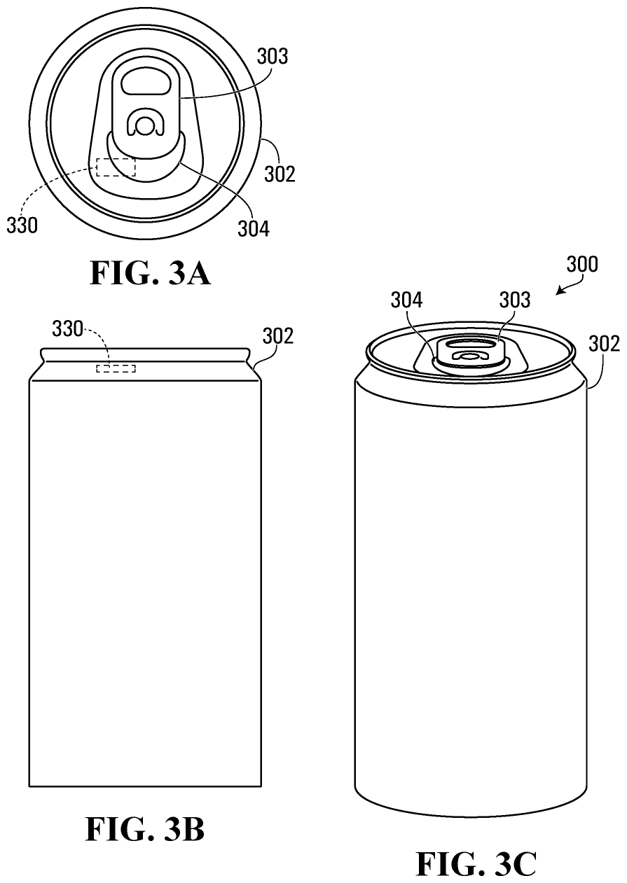

Containers could be other than bottle-type containers. FIGS. 3A to 3C are top, plan, and isometric views of a further example container, in the form of a can or tin-type container. The example container 300 includes a container body 302, and cover in the form of a part 304 of the top of the can. The part 304 is broken from a remainder of the can top or lid when the lift tab 303 is lifted away from the top of the can. Element 330 in FIGS. 3A and 3B generally represents a substance or device that could be released into the container body 302 when the container 300 is opened. For example, a nebulizer or gas release stimulant device could be sealed and/or otherwise attached to the underside of the lid and released from the lid when the part 304 is pushed toward the inside of the container body 302 by operating the lift tab 303. Opening of the container 300 could also or instead break a sealed packet or otherwise release an aerosolization substance and/or a gas release stimulant substance when the container is opened.

Some can or tin-type containers have covers in the form of pull tabs, and such containers could be used in other embodiments. Devices and/or substances could be sealed in some embodiments and attached to a pull tab, and released into a container body when the pull tab is removed to open a container.

An element such as a nebulizer or aerosolizer that is configured to be activated by opening a container may be used in conjunction with a can or tin type container 300 and activated when the container is opened, even though opening of such a container does not necessarily involve removing a cover.

The embodiments described above relate primarily to containers and their contents. Another aspect of the present disclosure relates to indicia or indicators that could be provided on containers and/or container packaging.

For example, in an embodiment a container that has a container body holding therein a cannabinoid-containing liquid for ingestion and an additive for production of a cannabinoid-containing aerosol for inhalation also includes one or more indicia or indicators. For example, an indicium may provide an estimate, or an indicator may be or include an indicator of an estimate, of effect of either or both of a cannabinoid-containing liquid and a cannabinoid-containing aerosol.

The effect could be or include, for example, a sober-up time from either or both of the cannabinoid-containing liquid and the cannabinoid-containing aerosol in some embodiments. Sober-up time may also be referred to as offset or offset time, and refers to how fast effects of the cannabinoid-containing liquid and/or aerosol are expected to wear off. Other examples of effects include an onset time of either or both of the cannabinoid-containing liquid and the cannabinoid-containing aerosol, and a maintenance time of either or both of the cannabinoid-containing liquid and the cannabinoid-containing aerosol. Onset refers to how quickly the cannabinoid-containing liquid and/or aerosol are expected to act or take effect. Maintenance refers to how long effects of the cannabinoid-containing liquid and/or aerosol are expected to act before wearing off. Maintenance and offset are not necessarily separately indicated. For example, onset may be measured from consumption to peak effect and then offset or maintenance may be measured from peak effect to a certain decline in effect.

Peak and/or decline may be defined, measured or specified in terms of cannabinoid level in the blood, for example. Other parameters to define, measure, or specify peak and/or other effects are also possible.

Indicia or indicators could include multiple estimates, represented as one or more plots in a graph for example, based on one or more user characteristics. The user characteristics could include, for example, any one or more of: body mass, gender, and age. Estimated effects could be plotted or otherwise represented versus time, for example. Plots for different body mass could use different line weights, with a heavier line weight corresponding to higher body weight and a lighter line weight corresponding to lower body weight, for example. Gender plots could use different colors, such as blue for male and pink for female. Different line patterns could be used to distinguish plots for different ages, such as dashes for a lower age and a line of “+” symbols for a higher age. These examples of plots and properties to distinguish between plots based on different user characteristics are intended for illustrative purposes only. Other indicia or indicators may be used in other embodiments.

A product package could include multiple containers. The containers in a product package could be the same, or the containers in a product package could include containers with different estimated effects of either or both of the cannabinoid-containing liquid and the cannabinoid-containing aerosol.

A product package, instead of or in addition to a container in such a package, could include one or more package indicia or package indicators providing an estimate of effect of either or both of the cannabinoid-containing liquid and the cannabinoid-containing aerosol in any or all containers. For example, for a product package that includes containers with different estimated effects, one or more package indicia or package indicators could be or include respective indicia or indicators providing an estimate of effect of either or both of the cannabinoid-containing liquid and the cannabinoid-containing aerosol in each container.

Such respective indicia or indicators may be on or otherwise associated with each of the containers. In some embodiments, one or more indicia or indicators of the different estimated effects are on or otherwise associated with the product package as a whole. Consider, for example, an embodiment in which indicia or indicators are provided on packaging such as a carton and on each container. Indicia or indicators on the packaging could then match or at least be consistent with an indicium or indicator of at least one container in the packaging. This could enable a user to, for example, match a current desired effect such as fast onset with an icon or other indicium on a carton, and then open the carton and select a container that carries the same icon or indicium.

Features that are disclosed by way of example herein with respect to container indicia or indicators may also or instead apply to package indicia or indicators. For example, package indicia or indicators may provide or be indicative of any of the example effects noted herein, and/or a package indicium or indicator may provide multiple estimates based on one or more user characteristics such as those noted above.

Indicia or indicators of effect are not limited to any particular type of container or product. For example, in some embodiments a cannabinoid-containing liquid or additive is aerosolizable by a nebulizer. An indicium or indicator in such an embodiment may include an indicium or indicator of non-use of the nebulizer associated with an estimate of effect of the cannabinoid-containing liquid only. The expected effect of the liquid is then apparent to a user, and the user may make a decision on that basis as to whether to consume only the cannabinoid-containing liquid, without any added effect, such as expected faster onset, of a cannabinoid-containing aerosol. An indicium or indicator may also or instead include an indicium or indicator of use of the nebulizer associated with an estimate of effect of the cannabinoid-containing aerosol only. A user can then decide whether to actually avail himself or herself of the expected added effect of using the nebulizer. Another possible option, instead of or in addition to liquid-only and/or aerosol-only indicia or indicators, is an indicium or indicator of use of the nebulizer associated with an estimate of effect of the cannabinoid-containing liquid in combination with the cannabinoid-containing aerosol, so that total expected effect of the liquid and aerosol is made readily apparent to the user.

Similarly, in embodiments in which a cannabinoid-containing liquid or additive is aerosolizable by addition of a further substance for production of the cannabinoid-containing aerosol, a container or package may include any of various indicia or indicators. These may include, for example, any one or more of: an indicator of non-use of the further substance associated with an estimate of effect of the cannabinoid-containing liquid only, an indicator of use of the further substance associated with an estimate of effect of the cannabinoid-containing aerosol only, and an indicator of use of the further substance associated with an estimate of effect of the cannabinoid-containing liquid in combination with the cannabinoid-containing aerosol.

In some embodiments, it may be possible for a user to controllably add amounts of a substance to produce the cannabinoid-containing aerosol. An indicium or indicator may include a respective indicium or indicator of each of a plurality of amounts of the further substance that can be controllably added for production of the cannabinoid-containing aerosol, associated with a respective estimate of effect the cannabinoid-containing aerosol produced by addition of each of the plurality of amounts of the further substance. As another possible option, an indicium or indicator may include a respective indicium or indicator of each of a plurality of amounts of the further substance that can be controllably added for production of the cannabinoid-containing aerosol, associated with a respective estimate of effect the cannabinoid-containing liquid in combination with the cannabinoid-containing aerosol produced by addition of each of the plurality of amounts of the further substance.

These examples may also apply to embodiments that include a gas release stimulant device or substance. For instance, an indicium or indicator may include any one or more of: an indicium or indicator of non-use of a gas release stimulant associated with an estimate of effect of the cannabinoid-containing liquid only, an indicium or indicator of use of the gas release stimulant associated with an estimate of effect of the cannabinoid-containing aerosol only, and an indicium or indicator of use of the gas release stimulant associated with an estimate of effect of the cannabinoid-containing liquid in combination with the cannabinoid-containing aerosol.

Indicia or indicators may also or instead be used in conjunction with dosage devices, examples of which are provided elsewhere herein. Consider, for example, a container that includes a container body holding a liquid; a cover releasably coupled to the container body to seal the container body; and a dosage device that includes a cannabinoid-containing substance, packaged with the container, to enable controlled addition of the cannabinoid-containing substance to the liquid to produce a cannabinoid-containing liquid for ingestion. Such a container may also include an indicium or indicator of any one or more of: an amount of the cannabinoid-containing substance to be released into the container and an estimated cannabinoid concentration in the cannabinoid-containing liquid. The amount and concentration referenced in this embodiment are other examples of characteristics or parameters for which one or more indicia or indicators may be provided. An indicium or indicator of an estimated effect may be provided in addition to or instead of indicia or indicators of amounts and/or concentrations, in dosage device embodiments or other embodiments.

Indicia or indicators need not be associated only with containers. For example, in some embodiments a product package includes covers. Each cover may be as disclosed by way of example elsewhere herein. One or more indicia or indicators may be especially useful in embodiments in which the covers include covers with dosage devices having one or more of: different cannabinoid-containing substances, different amounts of cannabinoid-containing substances, different estimated effects of cannabinoid-containing substances, and different granularity of control. A product package could include an indicium or indicator of an estimate of effect of either or both of the cannabinoid-containing liquid and the cannabinoid-containing substance for each cover. An indicium or indicator may also or instead include an indicium or indicator of any one or more of: an amount of the cannabinoid-containing substance to be released into the container by the dosage device, and an estimated cannabinoid concentration in the cannabinoid-containing liquid.

An indicium or indicator may include a respective indicium or indicator on each of the covers in a package.

In some embodiments, an indicium or indicator includes an indicium or indicator, on a product package, of the different estimated effects associated with different covers in the package.

Indicia or indicators may be applied to other embodiments, such as a combination that includes a container; a closure closing the container; a liquid, stored in the container, for ingestion; and a cannabinoid-containing gaseous substance, stored in the container, for inhalation when the closure closing the container is opened. In such an embodiment, the combination may also include an indicium or indicator of an estimate of effect of either or both of the cannabinoid-containing liquid and the cannabinoid-containing gaseous substance.

In the case of a product that includes a container; a liquid for ingestion; and a body of cannabinoid-containing gaseous substance for inhalation as a user drinks the liquid from the container, the product may also include an indicium or indicator, such as an indicium or indicator of an estimate of effect of the cannabinoid-containing gaseous substance. In some embodiments, the liquid is or includes a cannabinoid-containing liquid, and the cannabinoid-containing liquid and the cannabinoid-containing gaseous substance form a dual-phase cannabinoid-containing product. The indicium or indicator may provide an estimate of effect of the cannabinoid-containing liquid and the cannabinoid-containing gaseous substance.

Such indicia or indicators may be quite useful, and important, in helping users safely control or regulate their usage of products such as beverages, with or without delivery of one or more cannabinoids by aerosol. It is important to note that indicia or indicators need not require a high degree of interpretation by a user to ascribe meaning. Consider FIG. 1, for example. The very presence of the arrow 120 as an indicium or indicator on the cover 104 makes it readily apparent to a user that rotating the cover in the indicated direction, which is the direction in which the cover is normally rotated to tighten the cover, activates an additional feature or function.

In some embodiments, an icon such as a leaf or flower as referenced by way of example elsewhere herein, is also or instead provided. Such an icon next to or near a directional or instructional icon like the arrow 120, may provide an even clearer indication of how a cover or dosage device, for example, is to be operated or activated.

One or more dimensions of an indicium or indicator, such as the thickness of the arrow 120, may be increased in a direction of increasing effect. The arrow 120 may become wider from its tip to its tail, for example, to indicate that an effect increases as the cover 104 is turned in the direction shown. For example, a widening arrow may signify that more cannabinoid-containing substance is added to a container or liquid as the cover 104 is rotated in the clockwise direction in this example.

Regarding expected or estimated effect(s) of a cannabinoid-containing liquid and/or aerosol, any of various indicia or indicators may be used. One or more plots of expected or estimated effect over time may be used to provide an indication as to onset, offset, and/or maintenance time, and illustrative examples of plots are provided elsewhere herein. One or more range indicators of ranges and value indicators to indicate values within such ranges may also or instead be provided. For example, a line could be used as an indicator of a time period, with a marking to indicate time value. Consider onset time as an example. A line with an arrowhead pointing to the right could indicate an onset time range, and a marking along that line could indicate whether onset time for a cannabinoid-containing liquid and/or aerosol is relatively long or relatively short with respect to the total time duration.

As a simple example, consider a time line of the form “|------>” to indicate an onset time range, with “|” at the left indicating consumption by ingestion and/or inhalation for example and “>” indicating that the time line represents onset time. A short onset time could be indicated as “|-x----->”, whereas a longer onset time could be indicated as “|-----x->”. Maintenance times could be indicated similarly, with a marking with a range indicator “|------|” and the “|” symbols at both ends indicating that the timeline is a maintenance timeline, and/or offset time could use range/value indications with a range indicator “>------|” and the “>” symbol indicating that the timeline is an offset timeline. A clock icon could be used in combination with such range/value indicators, to indicate that the ranges/values relate to time. Other start/end/time indications are also possible, and these are just examples.

One or more clock, stopwatch, or time dial icons could also or instead be used to indicate time parameters. Such an icon could be used to indicate a time range, and a marker on or near an outline of the icon, on a time dial outline for example, could indicate a time value within the time range. For example, a dash or arrowhead at the “3 o'clock” position could indicate fast onset, or fast offset, or low maintenance time; a dash or arrowhead at the “6 o'clock” position could indicate an average or relatively longer onset, offset, or maintenance time; and a dash or arrowhead at the “9 o'clock” position could indicate slow onset, or slow offset, or long maintenance time. Several icons with respective markers could be used to indicate different times, or one icon with several different types of markers corresponding to different types of times such as onset, offset, and maintenance times could indicate several different times.

Range and value indications could also or instead be provided using “greyed out” range indicators and solid or darker value indicators. A time range could include five clock icons, for example, with relative time value within the range indicated by a number of solid or darker icons and remaining time range indicated by fainter or greyed out icons.

Multiple estimates based on one or more user characteristics, may be provided as noted above. Range indicators could be provided at various points along a reference pattern or plot, for example, to indicate how values are expected to vary with such characteristics. As another example, a reference plot based on average expected effect for a base set of characteristics could be provided in combination with plots of high and low expected variations of effect with different characteristics. High and low plots could instead be provided without a reference plot.

Several examples of devices or substances that may affect cannabinoid intake or availability, by producing aerosol and/or adding cannabinoid to a liquid for example, are disclosed herein. One or more indicia or indicators associated with use or non-use of such devices and/or substances, and or indicia or indicators associated with different amounts of substances, may be provided in some embodiments. In an embodiment in which a nebulizer or other device can but need not necessarily be activated or deployed, respective indicia of expected effect(s) could be provided next to or near respective icons. As an example, a liquid drop icon could be used to identify a liquid-only indicium or indicator of effect, a device or cloud icon could be used to identify an aerosol-only indicium or indicator of effect, and a combined icon such as a drop icon and a device icon with a “+” symbol, or even just a “+” symbol on its own, could be used to identify a combined liquid and aerosol indicium or indicator of effect.

Amount indicia or indicators could similarly denote corresponding indicia or indicators of estimated effects. A single line or the number “1”, for example, could indicate one dosage or amount of gas release stimulant, additive, or cannabinoid-containing substance, and identify an indicium or indicator of effect for that amount. Higher amounts could be indicated with an increasing number of lines or higher numbers. A cover that provides controllable addition of a substance in three amounts or portions, for example, could include one, two, and three strokes or other indicators, with adjacent or nearby indicators of corresponding effect(s).

These are just a few illustrative examples of indicia or indicators that may be provided in containers, covers, and/or packages. Others are also possible, including further examples provided herein.

Turning again to product embodiments, a drink producer, for example, could produce a product line including drinks of several different strengths, to provide a user with some choice as to preferred strength and/or expected effect. Although this might be practical for a certain number of different strengths, it might not be cost-effective or otherwise not feasible to maintain a product line with more than a certain number of different strengths. A product that provides a user with greater control over drink strength and/or expected effect might therefore be desirable.

According to an aspect of the present disclosure, a container or cover, for example, may be provided with a dosage device that allows a user to control how much of a cannabinoid-containing substance is added to a liquid in the container. A set, collection, or palette of covers that are different in some way, such as with different cannabinoid-containing substances, amounts, and/or estimated effects, could be packaged together to give a user even more choice over beverage content. For example, a “base” beverage could be sold in a bottle that is compatible with the set of covers and a user can then “cannabinoid-customize” the beverage by selecting a cover from the set. As another option, covers could be made to fit certain bottle sizes and/or types, and then any beverage in a compatible bottle can be customized with cannabinoid content according to user preference.

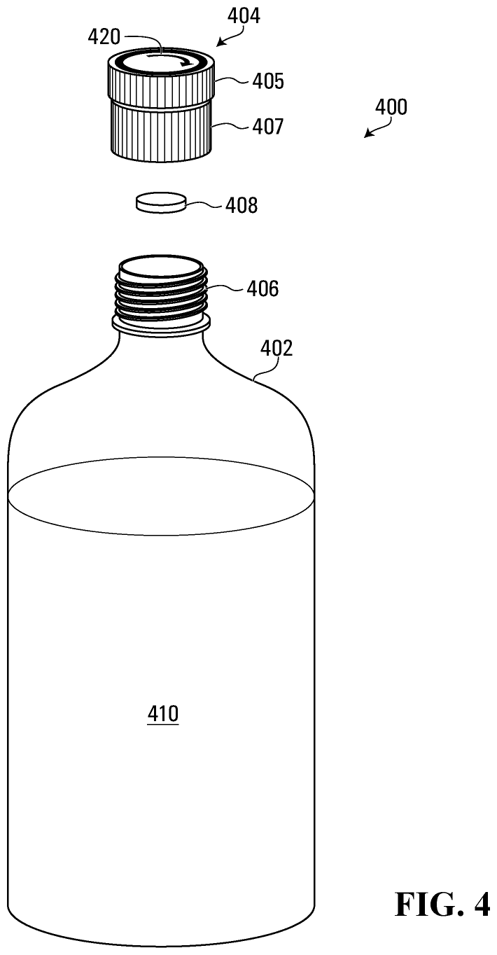

FIG. 4 is an exploded isometric view of another example container. The container 400 is substantially similar to the container 200 illustrated in FIG. 2, and includes a cover 404 which may be a multi-part cover with parts 405, 407 and/or may include an arrow and/or other operational indicator 420 in some embodiments. The indicator 420 may be an indicator of effect in other embodiments. A container may also or instead contain one or more other indicators of effect.

The container 400 also includes a container body 402 with external threads 406 on its neck to engage internal threads inside the cover 404. 410 represents a liquid for ingestion, which may or may not include a cannabinoid. 408 represents a dose of a cannabinoid-containing substance, which could be in liquid, tablet, powder, or other form.

The container 400 is illustrative of a container that includes a container body 402 holding a liquid 410 and a cover 404 that would be releasably coupled to the container body to seal the container body. According to an aspect of the present disclosure, a container also includes a dosage device comprising a cannabinoid-containing substance 408, packaged with the container, to enable controlled dosing or addition of the cannabinoid-containing substance 408 to the liquid 410 to produce a cannabinoid-containing liquid for ingestion.

In some embodiments, the cannabinoid-containing substance is or includes an emulsion. Such a substance may also be referred to as an emulsion system, an emulsification system, or an emulsification-based system, and examples are provided elsewhere herein.

The cannabinoid-containing substance may be or include a spray-dried emulsion, examples of which are also provided elsewhere herein. Spray drying may produce a powder that is ready to add to a liquid or container, for mixing with water or another liquid or beverage for example. A spray dried substance could be used in powder form, or tabletized to tablet form for example. In an embodiment, an emulsification-based system is provided in a cover or a cap. Such a cover or cap includes an emulsification-based system inside, for mixing with water or another liquid in a mixing chamber or a container, for example.

The dosage device is packaged inside the container body 402 in some embodiments, but could instead be externally packaged. Examples of a dosage device include a packet, capsule, or other component that contains an amount, also referred to herein as a dose or dosage, of a cannabinoid-containing substance; a device that is infused with, impregnated with, or otherwise includes a dose of a cannabinoid-containing substance; and one or more structures, in a cover for example, that contain a dose of a cannabinoid-containing substance. Other types of dosage devices are also possible.

The container 400 could be pressurized when sealed by the cover 404, in which case the dosage device could be pressure-sensitive and activated, by opening of the container, to release the cannabinoid-containing substance 408 into the container or directly into the liquid 410.

In some embodiments, the dosage device is carried by the cover 404. For example, the dosage device could be releasably carried by the cover 404 for release from the cover when the container 400 is to be opened.

A dosage device could be integrated with the cover 404, and be or include a compartment in the cover, for example. The dosage device may have a first state in which the cannabinoid-containing substance is held in the compartment and a second state in which at least a portion of the cannabinoid-containing substance is added to the liquid. The second state of the dosage device may enable fluid communication between the compartment and the container body. Examples of this are provided elsewhere herein.

In an embodiment, the cover 404 includes multiple parts 405, 407 as shown, and those parts define the compartment. The cover parts 405, 407 could be movable relative to each other, and such movement of the multiple parts relative to each other could control addition of the cannabinoid-containing substance 408 to the liquid 410 in some embodiments.

With a multi-part cover 404 having multiple parts 405, 407 that are movable between a number of positions relative to each other, the positions could be associated with respective amounts of the cannabinoid-containing substance 408 to be released into the container 400. This is one example of how a dosage device could be user-controllable to tailor drink strength or expected effect to user preference.

A dosage device may include a mixing chamber to enable mixing of the cannabinoid-containing substance and a portion of the liquid, and to inhibit mixing of the cannabinoid-containing substance and another portion of the liquid, as described in further detail by way of example elsewhere herein.

A dosage device could be “all-or-nothing” controllable to release either none of the cannabinoid-containing substance 408 or all of the cannabinoid-containing substance into the container 400 or the liquid 410. More granular control could be provided by a dosage device that is controllable to release none of the cannabinoid-containing substance 408, a portion of the cannabinoid-containing substance, or all of the cannabinoid-containing substance into the container 400 or the liquid 410.

In embodiments that provide for user-controlled dosing, it may be useful to implement a dosage device that includes one or more indicia or indicators of, for example, any one or more of: an amount of the cannabinoid-containing substance to be released into the container or liquid, and an estimated resultant cannabinoid concentration in the cannabinoid-containing liquid after the cannabinoid-containing substance has been released into the container or liquid.

For a turn-to-release embodiment in which turning the cover part 405 relative to the cover part 407 in the direction of the arrow 420 releases the cannabinoid-containing substance 408 into the container 400, for example, the indicia could be or include the arrow 420 as an indication of how the cover 404 is to be operated to add the cannabinoid-containing substance into the container. Text could also or instead be printed, formed, or otherwise affixed to the cover and/or elsewhere on the container 400, including on a container label, as an indicia to provide any of: instructions for operation, an amount of the cannabinoid-containing substance to be released into the container, and an estimated cannabinoid concentration in the cannabinoid-containing liquid. A dimension of the arrow 420 could increase, so that its width increases along its length from the head of the arrow to its tail for example, to provide an indication of increasing product release or liquid concentration as the cover part 405 is turned in the direction of the arrow. A dial or scale could also or instead be used as an indicium or indicator of an amount of substance to be added into a container or liquid from a dosage device. These are only examples, and other indicia or indicators such as symbols, icons, colors and/or other forms of visible and/or tactile indicia or indicators may be provided, in the embodiment shown and/or other embodiments. Other information could also or instead be provided in indicia on the cover 404 and/or elsewhere on the container 400, including on a label.

In some embodiments, a dosage device or another dosage device includes a further cannabinoid-containing substance, and is configured to enable controlled addition of the further cannabinoid-containing substance to the liquid. For example, in an embodiment the cannabinoid-containing substance comprises cannabidiol (CBD) and the further cannabinoid-containing substance comprises tetrahydrocannabinol (THC). A dosage device, or separate dosage devices, may release these substances separately. A cap could be turned one way to release CBD and/or turned an opposite way to release THC, for example. Another example would be a push/pull dispensing or release mechanism to release one substance on a pull operation and another substance on a push operation. One or more indicia or indicators of the substance(s), amount(s), estimated effect(s), and/or operating instructions may be provided, in or on a cover, container, or label for example.

In the context of a cannabinoid-containing liquid 410 or other cannabinoid-containing substance such as 408, it may be desirable to restrict access to the contents of a container. For example, the cover 404 could include one or more child-resistant features to restrict access to contents of the container 400. Child-resistant features could also or instead be implemented in other containers disclosed by way of example herein, whether such containers hold pre-mixed cannabinoid-containing liquid or provide for user controlled dosing in mixing a cannabinoid-containing liquid as in FIG. 4.

In some embodiments, the dosage device also or instead includes one or more child-resistant features to restrict access to the cannabinoid-containing substance 408.

Other features may also or instead be provided. For example, a cover may include a mouthpiece to enable drinking from the container.

Various container and cover features are disclosed generally by way of example above. Illustrative examples of a cover are now considered in more detail.

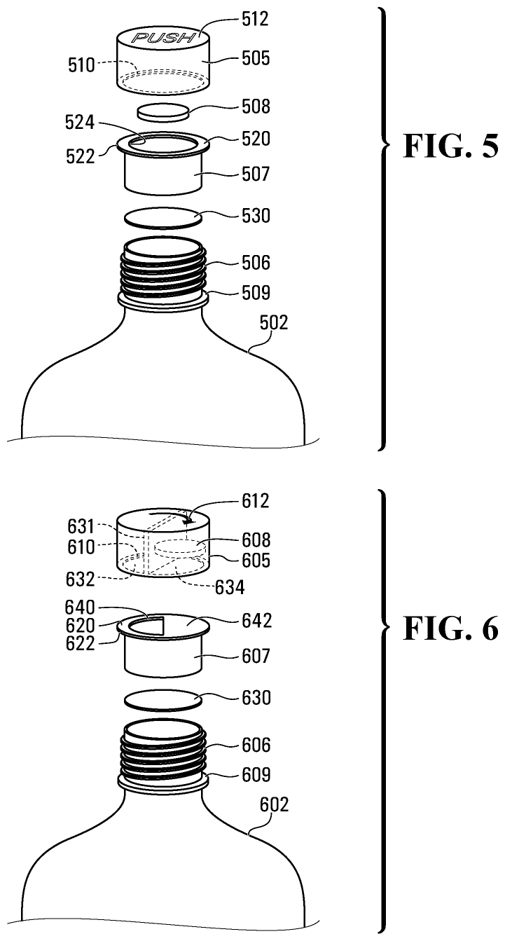

FIG. 5 is an exploded isometric view of a cover according to another embodiment. The example cover includes multiple cover parts 505, 507 and a seal 530. The seal 530 is held against the top of container body 502 by an inner part 524 of a shoulder or flange 520 on the cover part 507, to seal the container body, when the cover part 507 is screwed onto the neck of the container body. The example in FIG. 5 illustrates a threaded connection between the container body 502 and the cover part 507, in which outer threads 506 on the container body neck engage inner threads in the cover part 507. Element 509 is a shoulder on the neck of the container body 502.

The cover parts 505, 507 would be press-fit together in the example shown, by forcing an inner shoulder or flange 510 inside the cover part 505 over the outer part 522 of the flange 520 on the cover part 507. The cannabinoid-containing substance 508 could be placed on the seal 530 or inside the cover part 505 before the cover parts 505, 507 are coupled together, and the cannabinoid-containing substance is then sealed inside the cover, between the seal and the cover part 505 in the example shown. The cover parts 505, 507 could instead be coupled together, and the cannabinoid-containing substance 508 could then be added into the cover before the seal 530 is put into place in the cover. In another embodiment, the cover parts 505, 507 are assembled, the seal 530 is placed on top of the container 502, and the cannabinoid-containing substance 508 is placed either into the cover or onto the seal 530 before the cover is screwed onto the neck of the container.

When the cannabinoid-containing substance 508 is to be released into the container body 502 in the example shown, the cover part 505 is pushed axially toward and over the cover part 507 to break the seal 507 and release the cannabinoid-containing substance into the container body 502. The indicium 512 in FIG. 5 is an illustrative example of an indicium or indicator that could be provided to indicate to a user how to release the cannabinoid-containing substance 508 into the container body 502. Although the indicium 512 is text in the example shown, other indicia or indicators, including icons such as a leaf or flower icon and/or one or more arrows on the outer wall of the cover part 505 pointing downward for example, are possible. One or more indicia or indicators of other properties or characteristics may also or instead be provided, as disclosed by way of example elsewhere herein.

In another embodiment, the cannabinoid-containing substance 508 could be released from the cover part 505 by rotating the cover part 507 in a direction to over-tighten the cover part 507 and break the seal 530 along its edge, where it contacts the top of the container body 502. This could itself release the cannabinoid-containing substance 508 from the cover part 505 into the container body 502, or the cover part 505 could be pushed as described above after the seal 530 has been broken. An indicium or indicator could be provided on the cover part 507, the cover part 505, and/or elsewhere on a container including on a label, to indicate to a user that release of the cannabinoid-containing substance 508 into the container body 502 involves overtightening the cover part 507 and then pushing the cover part 505.

FIG. 6 is an exploded isometric view of another example cover. The example cover includes multiple cover parts 605, 607 and a seal 630. The seal 630 is held against the top of container body 602 by an upper disc or wall 620 on the cover part 607, to seal the container body, when the cover part 607 is screwed onto the neck of the container body by engaging outer threads 606 on the container body neck with inner threads in the cover part 607. Element 609, like element 509, is a shoulder on the neck of the container body 602.

As in FIG. 5, FIG. 6 illustrates cover parts 605, 607 that would be press-fit together by forcing an inner shoulder or flange 610 inside the cover part 605 over an outer part 622 of a flange or shoulder on the cover part 607. The flange or shoulder on the cover part 607 could be formed integrally with the disc or wall 620, or separately. The disc 620 includes an aperture 640 and a closed portion 642.

Inside the cover part 607, an internal wall 631 divides the cover part interior space into separate compartments 632, 634. The internal wall 631 could extend from a top wall and along the cylindrical side wall of the cover part 607 in the example shown. In order to provide clearance for press-fitting the cover parts 605, 607 together, the internal wall 631 does not extend all the way to the internal shoulder or flange 610.

The cannabinoid-containing substance 608 could be placed on the closed portion 642 of the wall 620 or inside compartment 608 before the cover parts 605, 607 are coupled together, with the closed portion 642 aligned with the compartment 634 to enclose the cannabinoid-containing substance inside the cover.

When the cannabinoid-containing substance 608 is to be released into the container body 602, the cover part 605 is rotated, in the direction indicated by arrow 612 in the example shown, to at least partially align the compartment 634 with the aperture 640. The cannabinoid-containing substance 608 can be released into the container body 602 when the seal 630 is broken, illustratively by over-tightening as described above.

In another embodiment, a seal between the top of the container body 602 and the cover part 607 is open at least in the area of the aperture 640. For example, and O-ring or washer type seal could be used instead of the seal 630, and the cannabinoid-containing substance 608 is released into the container body 602 when the compartment 634 is aligned with the aperture 640 sufficiently for the cannabinoid-containing substance 608 to pass through the aperture. In another embodiment, a cover part 507 as shown in FIG. 5 is used, and the seal includes an aperture. In embodiments with an open or apertured seal, additional seals or structural features could be provided to seal between the cover parts 605, 607, and to seal the compartment 634 from the interior of the container body 602.

FIG. 6 illustrates an example of a cover that could be used to implement an “align to release” mechanism. One or more indicia or indicators such as alignment marks or arrows could be provided on the cover parts 605, 607 to indicate alignment for release of the cannabinoid-containing substance 608 into the container body 602. Physical features such as one or more detents and projections could be provided in or on the cover parts 605, 607, to limit relative movement of the cover parts between non-release and release positions, and/or to provide feedback to a user so that the user can detect when the cover parts have been properly aligned to release the cannabinoid-containing substance.

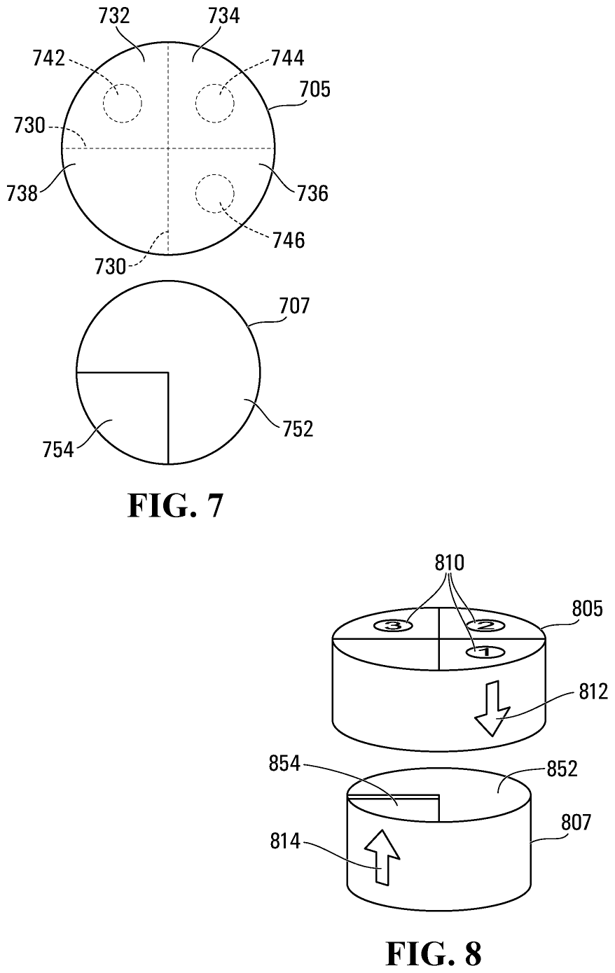

Alignment-based release could be extended to provide more granular dosing control, as shown by way of example in FIG. 7, which includes top views of cover parts of a further example cover.

In FIG. 7, multiple internal walls 730 partition the inside of a cover part 705 into multiple compartments 732, 734, 736, 738, three of which include cannabinoid-containing substance doses 742, 744, 746. A cover part 707 has a top wall with a closed portion 752 and an aperture 754. The cover parts 705, 707 could be operated in much the same way as the cover parts 605, 607 in FIG. 6, to release one or more of the cannabinoid-containing substance doses 742, 744, 746, one at a time, into a container body. This could be useful in providing a user with more granular dosing control than a single-dose mechanism as in FIG. 6. Physical features to limit relative movement of the cover parts 705, 707 between a non-release position and the multiple release positions and/or to provide feedback to a user regarding movement between positions could be especially useful in a multi-dose embodiment such as FIG. 7. For example, the cover parts 705, 707 could include structural features to releasably hold the cover parts in each position, so that a user could turn the cover part 705 to “click” once to release dose 746, optionally again to also release dose 744, and optionally once more to also release dose 742. This type of feedback is illustrative of tactile feedback or a tactile indicator that may be provided in some embodiments.

Other embodiments could include more or fewer compartments, multiple apertures, and/or compartments or apertures with different shapes than shown. Also, although the compartments and apertures in FIGS. 6 and 7 have corresponding shapes, in other embodiments an aperture need not have the same shape as a compartment. A cannabinoid-containing substance dose could be in powder or liquid form, for example, and could be stored in a compartment having one shape and flow into a container body through an aperture that has a different shape.

FIG. 8 is an exploded isometric view of yet another example cover. Although shown and labelled differently from FIG. 7, the example cover in FIG. 8 illustrates indicia that could be provided form a multi-dose cover as shown in FIG. 7.

In FIG. 8, the indicia at 810 on cover part 805 provide a user with an indication that there are three separate doses of cannabinoid-containing substance available for release into a container body. Dose information, which could be different for each dose, could also or instead be provided. An arrow outside each compartment on cover part 805, one of which is shown at 812, and an arrow 814 on cover part 807 are illustrative of indicia that could be used to provide a user with an indication as to alignments to release each cannabinoid-containing substance dose into the container body. The cover part 807 has a top wall with a closed portion 852, and an aperture 854 through which one or more of the three doses of cannabinoid-containing substance may be introduced into a container when the arrow 812 outside each compartment of cover part 805 is aligned with the arrow 814.

The example covers in FIGS. 5 to 8 all include a coupling structure, in the cover parts 507, 607, 707, 807, to releasably couple the cover to a liquid container, a seal to seal the container, and a dosage device that includes a cannabinoid-containing substance, to enable controlled addition of the cannabinoid-containing substance to a liquid inside the container to produce a cannabinoid-containing liquid for ingestion.

The dosage device is integrated with the covers in these examples, and in each example the dosage device includes a compartment in the cover. There is only one compartment in the example shown in FIG. 5, and there are multiple compartments in the other examples.

These example covers all include multiple parts that define the compartment and are movable relative to each other, and movement of the multiple parts relative to each other controls addition of the cannabinoid-containing substance to the liquid. The cover parts are movable between a number of positions relative to each other, and the positions are associated with respective amounts of the cannabinoid-containing substance to be released into the container. In FIG. 5, the dosage device is controllable to release either none of the cannabinoid-containing substance 508 or all of the cannabinoid-containing substance into the container, whereas the other example covers provide more granular control. The example covers in FIGS. 6 to 8 include a dosage device that is controllable to release none of a cannabinoid-containing substance, a portion of the cannabinoid-containing substance, or all of the cannabinoid-containing substance into the container.

A dosage device could include one or more indicia of an amount of the cannabinoid-containing substance to be released into the container and/or an estimated cannabinoid concentration in the cannabinoid-containing liquid, for example. FIG. 8 illustrates an example at 810. Other indicia or indicators, such as any of those disclosed by way of example herein, may also or instead be provided.

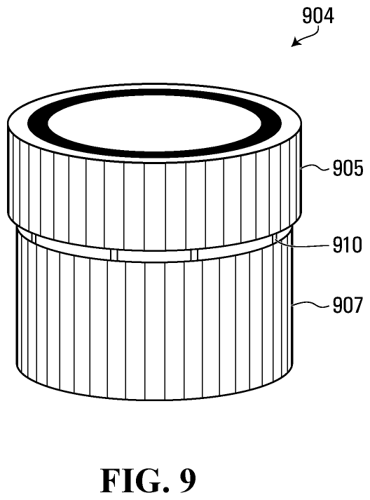

Although not shown in FIGS. 5 to 8, a multi-part cover could include additional features to reduce the likelihood of inadvertent release of a cannabinoid-containing substance into a container body. FIG. 9 is an isometric view of another example cover 904 in which cover parts 905, 907 are coupled together by a breakable coupling, in the form of segments or interconnections 910. In order to release the cannabinoid-containing substance by moving one cover part 905, 907 relative to the other, by turning or pushing on the cover part 905 for example, the interconnections 910 must be broken. In a turn-to-release embodiment, the cover part 905 could be turned in a direction that might over-tighten the cover. Rotation of the cover part 907 could be limited by a stop such as a shoulder on the neck of a container body, as shown at 509, 609 in FIGS. 5 and 6. In such an embodiment, the cover part 905 would be rotatable relative to the cover part 907 to break the interconnections 910 at least when the cover part 907 abuts the shoulder.

Also, as noted elsewhere herein, a cover and/or dosage device could include one or more child-resistant features to restrict access to contents of the container. A cover or dosage device could include a push-to-turn or align-to-remove cover or cover part, for example. With reference to FIG. 9, a breakable coupling could also or instead provide a certain degree of child resistance or access restriction. A cover having a basic structure similar to the example shown in FIG. 9 is not necessarily restricted to implementation with an integrated dosage device. For example, a similar cover could secure a container in a sealed state with a band at 907 positioned below a shoulder on the neck of a container body, as shown at 509, 609 in FIGS. 5 and 6, and attached to a threaded cover at 905 by interconnections 910. In order to open the container, the interconnections 910 would have to be broken.

Dosage devices need not be carried by or integrated with container covers. A pressure-sensitive dosage device, for example, could be placed into a container that is pressurized when sealed, and activated by opening of the container to release the cannabinoid-containing substance into the container or liquid.

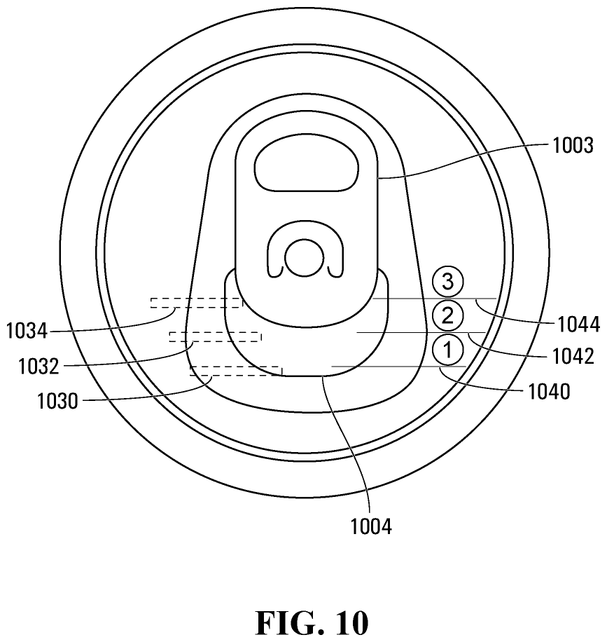

Containers with dosing control are not limited only to bottle-type containers. FIG. 10 is a top view of an example container with dosing control, in particular a can or tin-type container. The example container in FIG. 10 includes a container body 1002, and a cover in the form of a part of the top of the can, labeled as 1004, which is broken from a remainder of the can top or lid when the lift tab 1003 is lifted away from the top of the can. Elements 1030, 1032, 1034 in FIG. 10 represent a dosage device, such as a sealed packet or capsule that could be ruptured, to release a controlled amount of a cannabinoid-containing substance into the container body 1002 when the container is opened. Dosing control in this example is based on how far the can is opened, and indicia at 1040, 1042, 1044 indicate to a user how far the can should be opened to release each of three doses of cannabinoid-containing substance into the container body 1002. Although numbers are shown in FIG. 10, icons or other indicia or indicators, such as just the illustrated lines, are used in other embodiments.

Similar dosing control could be provided for can or tin-type containers with pull tabs, with indicia to delineate how far a pull tab should be removed to release each of one or more doses into a container or liquid.

Other features may be provided in conjunction with embodiments described with reference to FIGS. 5 to 10, and non-limiting examples of such features are noted below.

For example, a cannabinoid-containing substance in such embodiments may be or include an emulsion, such as a spray-dried emulsion. Although covers with integrated dosage devices are shown FIGS. 5 to 8, dosage devices that are instead releasably carried by the cover for release from the cover when the container is to be opened are employed in other embodiments.

Multi-state dosage devices are used in some embodiments, and illustrative examples of first and second stages of such dosage devices are provided elsewhere herein. A dosage device with a mixing chamber may also or instead be provided, and an example of such a dosage device is also provided elsewhere herein.

A dosage device, or another dosage device, may include a further cannabinoid-containing substance and be configured to enable controlled addition of the further cannabinoid-containing substance to the liquid in a container. Illustrative examples of such features are also provided herein, at least above.

A cover and/or a dosage device may include one or more child-resistant features in any of the embodiments described with reference to FIGS. 5 to 10. A mouthpiece to enable drinking from a container is another feature that is described by way of example elsewhere herein and may be implemented in such embodiments.

Covers need not be sold only, or even at all, with containers. For example, a product package could include multiple covers which a user could attach to a drink container in order to mix a cannabinoid-containing liquid for ingestion. The covers in a package could include covers with dosage devices having one or more of: different cannabinoid-containing substances, different amounts of cannabinoid-containing substances, different estimated effects of cannabinoid-containing substances, and/or different granularity of dosing control, for example.

Other cover features disclosed by way of example herein may be implemented in cover package embodiments. For example, the covers in a package may include a cover for a container that is pressurized when sealed by the cover and a dosage device that is pressure-sensitive and is activated, by opening of the container, to release the cannabinoid-containing substance into the container.

As another example, the covers in a package may also or instead include a cover with a dosage device that is releasably carried by the cover for release from the cover when the container is to be opened. A cover with a dosage device that includes a compartment in the cover may also or instead be provided in a package. Such a cover may include multiple parts that define the compartment and are movable relative to each other, for example. Movement of the multiple parts relative to each other controls addition of the cannabinoid-containing substance to the liquid in some embodiments. For example, the cover parts may be movable between multiple positions relative to each other, and the positions are associated with respective amounts of the cannabinoid-containing substance to be released into the container or liquid.

The covers in a package may include a cover with a dosage device that is controllable to release either none of the cannabinoid-containing substance or all of the cannabinoid-containing substance into the container or liquid, and/or a cover with a dosage device that is controllable to release none of the cannabinoid-containing substance, a portion of the cannabinoid-containing substance, or all of the cannabinoid-containing substance into the container or liquid. In such embodiments, a package may also include one or more indicia or indicators of any one or more of: an amount of the cannabinoid-containing substance to be released into the container or liquid by the dosage device; and an estimated cannabinoid concentration in the cannabinoid-containing liquid. Indicia or indicators of one or more other characteristics or parameters may also or instead be provided.

One or more covers in a package may include a child-resistant feature to restrict access to contents of the container. The covers in a package may include one or more covers with a dosage device that has a child-resistant feature to restrict access to the cannabinoid-containing substance.

Each cover or dosage device, and/or a product package, could include one or more indicia or indicators such as a label, legend, color coding, etc., providing information about each cover or dosage device. A product package may include an indicium or indicator of an estimate of effect of either or both of a cannabinoid-containing liquid and a cannabinoid-containing substance, for example. The indicium or indicator may include a respective indicator on each of the covers. More generally, a respective indicium or indicator could be provided on each of the covers, and/or an indicia could be provided on the product package, to provide information about any one or more of: different products or substances, different amounts, different estimated effects, and the different granularity of control associated with each cover, for example.

Embodiments disclosed herein could provide a combination that includes a container; a closure, also referred to herein as a cover, closing the container; and a liquid, stored in the container, for ingestion. The liquid could be or include a cannabinoid-containing liquid, and could be a pre-mixed or prepared liquid that already includes a cannabinoid when the liquid is packaged into the container, or a liquid into which a cannabinoid-containing substance is to be added by a user. Such a combination could also include a cannabinoid-containing gaseous substance, stored in the container, for inhalation when the closure closing the container is opened. A cannabinoid-containing gaseous substance is also referred to herein as a cannabinoid-containing aerosol, and could be produced from contents of the container and/or added to and sealed into the container with the liquid.

Also consistent with the present disclosure, a product could include at least a container and a liquid for ingestion. A body of cannabinoid-containing gaseous substance could also be in the container, above the liquid for example, for inhalation as a user drinks the liquid from the container. The gaseous substance need not necessarily be disposed above the liquid, and could be located beside the liquid, in a separate compartment inside a container for example.

In these combination and product examples, and other embodiments disclosed herein, a cannabinoid-containing liquid and a cannabinoid-containing gaseous substance could be considered a form of dual-phase cannabinoid-containing product.

Other features disclosed herein could be applied to these combination and product examples. For example, the cannabinoid-containing liquid and the cannabinoid-containing gaseous substance could include the same cannabinoid(s) or different cannabinoids. The cannabinoid-containing liquid and the cannabinoid-containing gaseous substance could also or instead have different cannabinoid concentrations. The cannabinoid-containing gaseous substance could have a higher cannabinoid concentration than the cannabinoid-containing liquid, for example. A container or product could include one or more indicia or indicators providing an estimate of effect of either or both of the cannabinoid-containing liquid and the cannabinoid-containing gaseous substance. These are illustrative and non-limiting examples of additional features that are disclosed herein and could be applied to combinations or products that include a liquid and a cannabinoid-containing gaseous substance

Embodiments described above relate primarily to liquid formulations and containers. Other embodiments, including methods, are also contemplated.

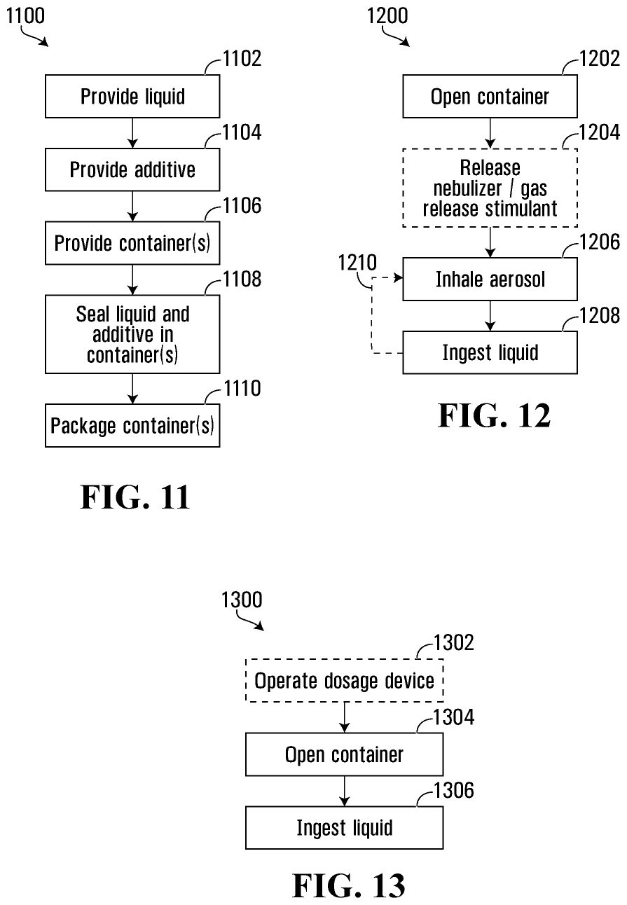

FIG. 11, for example, is a flow diagram illustrating a method for production of a cannabinoid-containing product. The example method 1100 involves an operation 1102 of providing a cannabinoid-containing liquid for ingestion, and an operation 1104 of providing an additive for production of a cannabinoid-containing aerosol for inhalation. These operations 1102, 1104 are shown separately for illustrative purposes, but need not be separate operations in all embodiments. For example, an additive such as dissolved gas could be integrated into a cannabinoid-containing liquid, in which case the cannabinoid-containing liquid and the additive are provided together.

Either or both of the cannabinoid-containing liquid and the additive could be provided at 1102, 1104 by actually preparing the liquid and/or additive. The cannabinoid-containing liquid and/or the additive could instead be provided at 1102, 1104 by purchasing or otherwise acquiring the liquid and/or additive from one or more suppliers.

Similarly, one or more containers could be provided at 1106 by manufacture, or through purchase or other acquisition. Different container parts, such as container bodies and covers, could be provided at 1106 in different ways, for example by manufacturing one container part and purchasing another container part or by purchasing different container parts from different suppliers. In general, one or more containers could be provided at 1106. In an industrial application it is likely that multiple containers would be provided at 1106 and have the liquid and additive sealed inside at 1108 in a production run. Sealed containers are then packaged at 1110 in an embodiment.

The example method 1100 is illustrative of one embodiment. Examples of various ways to perform the illustrated operations, additional operations that may be performed in some embodiments, or operations that could be omitted in some embodiments, could be inferred or apparent from the description and drawings relating to liquid formulations, containers, and covers, for example. Further variations may be or become apparent.

For instance, any of various types of liquids and/or additives could be provided at 1102, 1104, a nebulizer and/or a gas release stimulant could be provided and packaged with or inside containers, and containers could be provided with any of various features as disclosed by way of example elsewhere herein.

Some embodiments disclosed herein relate to a container that includes a container body holding contents that include a liquid for ingestion, a cover releasably coupled to the container body to seal the container body, and an aerosolizer packaged with the container to aerosolize a cannabinoid from the contents for airborne delivery of a cannabinoid-containing aerosol for inhalation. A method of producing a container of this type could be substantially similar to the example method 1100, with the exception that an aerosolizer would be provided at 1104 and optionally sealed in a container at 1108, instead of or in addition to an additive.

Methods related to a dosage device could also be substantially similar to the example method 1100, but with a dosage device, instead of or in addition to an additive, being provided at 1104 and optionally sealed in a container at 1108.

An embodiment of a method related to a dosing device involves providing a cannabinoid-containing substance and adding the cannabinoid-containing substance to a cover that includes a coupling structure to releasably couple the cover to a liquid container; a seal to seal the container; and a dosage device to enable controlled addition of the cannabinoid-containing substance to a liquid inside the container to produce a cannabinoid-containing liquid for ingestion. The cannabinoid-containing substance is or includes an emulsified cannabinoid-containing substance in some embodiments. The substance is a spray dried cannabinoid-containing substance in some embodiments.

In some embodiments, a combination or product includes a liquid for ingestion and a cannabinoid-containing gaseous substance for inhalation. The example method 1100 relates to production of such a gaseous substance from an additive. A cannabinoid-containing gaseous substance could also or instead be added to and seal in a container with a liquid, which might or might not be a cannabinoid-containing liquid. In a method to produce a combination or product with a liquid and cannabinoid-containing gaseous substance in a container, the cannabinoid-containing gaseous substance could be provided at 104 instead of or in addition to the additive, and sealed in the container with at least the liquid at 1108. The cannabinoid-containing gaseous substance could be pumped into or otherwise added to a container before, after, or during addition of the liquid into the container, and the container could then be sealed.

Thus, a container might or might not include a cannabinoid-containing gaseous substance when it is filled and sealed. A cannabinoid-containing gaseous substance could be added to and sealed in the container or produced after the container is sealed. In either case, a sealed container in these examples includes a liquid for ingestion, which could but need not necessarily contain a cannabinoid, and a cannabinoid-containing gaseous substance or aerosol for inhalation after the container is opened.

Even an open container could include a cannabinoid-containing gaseous substance for inhalation. In some embodiments, aerosol production could be stimulated or initiated multiple times during consumption of the liquid after the container has been opened. Unless all of a cannabinoid-containing gaseous substance that is in a container exits the container after the container is opened, then at least some of the cannabinoid-containing gaseous substance would remain present in the open container and available for inhalation by the user. Again, the cannabinoid-containing gaseous substance could have been added to and sealed in the container or subsequently produced from contents of the container.

Regarding the operation of providing the container(s) at 1106, this could involve providing particular types of covers, such as covers that include a dosage device. Cover parts including a coupling structure to releasably couple the cover to a liquid container and a seal to seal the container could be provided with a dosage device, and used to seal one or more containers at 1110.

Other variations of methods associated with producing a cannabinoid-containing product may be or become apparent.

User methods are also contemplated. FIG. 12 is a flow diagram illustrating a method of use according to an embodiment. The example method 1200 involves an operation 1202 of opening the container, an optional operation 1204 in which a nebulizer, a gas release stimulant, and/or or another device or substance to produce an aerosol is released in some embodiments, inhaling a cannabinoid-containing aerosol at 1206, and ingesting a cannabinoid-containing liquid at 1208.

Variations in the method 1200 could include, for example, releasing a nebulizer and/or gas release stimulant before opening the container. In some embodiments, the aerosol is produced without any such release of a device or substance into the container. A container or product could already include a cannabinoid-containing gaseous substance for inhalation.

The dashed line at 1210 represents another variation in which aerosol is not necessarily inhaled only before the liquid is ingested at 1208. This could involve multiple releases at 1204. An aerosol could be inhaled multiple times before a liquid is completely consumed. It is also possible that some liquid could be consumed before an initial aerosol inhalation.

Although shown separately at 1206, 1208, inhalation and ingestion could be performed at substantially the same time. For example, a container could store a body of cannabinoid-containing gaseous substance above a liquid, for inhalation as a user drinks the liquid from the container.

FIG. 13 is a flow diagram illustrating a method of use according to another embodiment. In the example method 1300, a dosage device is operated at 1302 to add a controlled amount of cannabinoid-containing substance to a liquid, to produce a cannabinoid-containing liquid. The container is opened by the user at 1304, and the cannabinoid-containing liquid is ingested at 1306.

Operation 1302 is shown in dashed lines to represent the fact that operating a dosage device could be integrated with or automatically performed with opening a container at 1302. A packet or capsule containing a dose of cannabinoid-containing substance could be ruptured when a container is opened, for example, to thereby operate the dosage device when the container is opened.

A dosage device could instead be operated after a container is opened at 1304.

Opening of a container at 1304 is also potentially optional. For example, a mouthpiece may be provided to enable drinking from the container without fully opening the container.

Other operations could also be performed. For example, after the cannabinoid-containing substance is added to the liquid, the product and liquid could be mixed. Mixing could involve agitating the container contents, before and/or after the container is opened.

The example methods 1200, 1300, like the example method 1100, are illustrative and non-limiting examples. Various ways to perform the illustrated operations, additional operations that may be performed in some embodiments, or operations that could be omitted in some embodiments, could be inferred or apparent from the description and drawings relating to liquid formulations, containers, and covers, for example, or otherwise be or become apparent.

Other features may also or instead be provided in some embodiments. For example, a user might desire control over the quantity of cannabinoid-containing liquid that is produced using a dosage device, illustratively by adding a cannabinoid-containing substance to only a portion of the liquid inside of a container, rather than all of the liquid. Only this portion of the liquid could be used to produce an amount of cannabinoid-containing liquid for consumption by the user. The user could then consume any or all of the remainder of the liquid with little or no cannabinoids.

Furthermore, a user might not want to add a cannabinoid-containing substance directly into the body of a container, as this could contaminate the inside of the container. This could be relevant for reusable containers, for example. Adding a cannabinoid-containing substance directly into a reusable container could potentially affect future uses of the container, and/or require a user to wash the container to remove any traces of the cannabinoid-containing substance before using the container again.

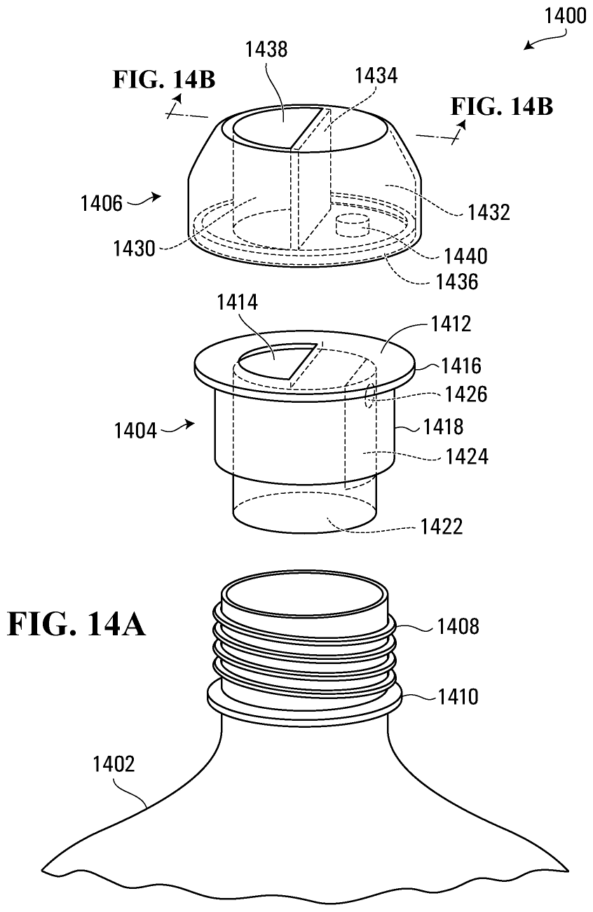

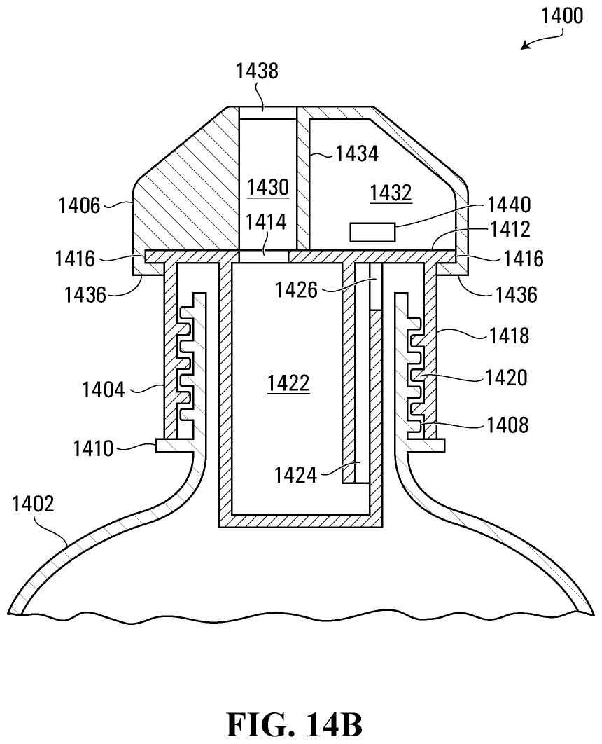

In some embodiments, a dosage device includes a mixing chamber to enable mixing of a cannabinoid-containing substance and a portion of a liquid, and to inhibit mixing of the cannabinoid-containing substance and another portion of the liquid. Optionally, the mixing chamber could inhibit movement of the cannabinoid-containing substance into a container body. In some embodiments, a mixing chamber is an example of an intermediate chamber that can separate a portion of the liquid from a container body, and mix this portion of the liquid with a cannabinoid-containing substance to produce a cannabinoid-containing liquid. The mixing chamber can then maintain separation of the cannabinoid-containing liquid from the container body itself. An example of a mixing chamber is described below with reference to FIGS. 14A and 14B.

FIG. 14A is an exploded isometric view of another example container 1400, and FIG. 14B is a cross-sectional assembled view of the container 1400, taken along the line 14B--14B in FIG. 14A. The container 1400 includes a container body 1402 and multiple cover parts 1404, 1406.

The container body 1402 includes a neck with threads 1408 and a shoulder or flange 1410. The cover part 1404 includes a surface 1412 with a hole or opening 1414 therein, a shoulder or flange 1416, a cylindrical side wall 1418, threads 1420 extending radially inwardly from the side wall 1418, a mixing chamber 1422, a passage or channel 1424, and an a hole or opening 1426 in a wall that forms part of the channel 1424. The cover part 1406 includes a passage or channel 1430, a compartment 1432, an internal wall 1434, a shoulder or flange 1436, and a hole or opening 1438 connected to the channel 1430. A cannabinoid-containing substance 1440, which could be in liquid, tablet, powder, or other form, is illustrated inside of the compartment 1432.

In some implementations, the container body 1402 is similar to any one of the container bodies 102, 202, 402, 502, 602, for example. The container body 1402 could be a disposable container or a reusable container.

Each of the cover parts 1404, 1406 could be integrally formed from a single material through a machining, molding and/or extruding process. Alternatively, either or both of the cover parts 1404, 1406 could be formed from one or more parts that are coupled together using adhesives and/or fasteners, for example. Example materials for a container body such as 1402 and cover parts such as 1404, 1406 are provided elsewhere herein.

FIG. 14B illustrates the container 1400 in an assembled state. During assembly, the cannabinoid-containing substance 1440 is placed within the compartment 1432, and the cover part 1406 is press-fit over the cover part 1404. During press-fitting, the flange 1436 could be forced over the flange 1416. In the illustrated example, a top surface of the flange 1436 abuts a bottom surface of the flange 1416. The internal wall 1434 does not extend all of the way to the flange 1436 in order to provide clearance for press-fitting. The internal wall 1434 abuts the surface 1412 following assembly of the cover parts 1404, 1406.

In some implementations, one or more gaskets and/or seals are also provided. For example, one or more gaskets or seals could seal the channel 1430 and/or compartment 1432, with the possible exception of the openings 1414, 1438. In one example, a ring-like seal is disposed at the abutment between the flanges 1416, 1436 to inhibit the flow of liquid between these flanges. In another example, a disk-like seal is disposed on the surface 1412 to seal the perimeter of the channel 1430. The disk-like seal could include an opening that is adjacent and complementary to the opening 1414. The compartment 1432 could also or instead be sealed using one or more seals or gaskets.

The threads 1420 on the side wall 1418 are complementary to the threads 1408 on the neck of the container body 1402. As such, the cover part 1404 can be screwed onto the container body 1402. One or more gaskets or seals could be disposed between the container body 1402 and the cover part 1404 to seal the perimeter of the neck of the container. In some implementations, the cover part 1404 includes one or more child-resistant features in order to restrict access to contents of the container body 1402 and/or to the contents the cover parts 1404, 1406.

In the assembled state illustrated in FIG. 14B, the channel 1430, the mixing chamber 1422, and the interior space of the container body 1402 are in fluid communication. Specifically, liquid from the container body 1402 can enter the cover part 1404 through the opening 1426, and then flow through the channel 1424 and into the mixing chamber 1422. From the mixing chamber 1422, the liquid can flow into the channel 1430 through the opening 1414, and then out of the channel 1430 through the opening 1438. As such, liquid from the container body 1402 can be consumed by a user through the opening 1438. At least in this sense, the cover part 1406 could be considered an example of a mouthpiece. In some implementations, the container body 1402 is deformable, and a user can squeeze the container body to force liquid through the cover parts 1404, 1406. In some implementations, the opening 1438 and/or other portions of the cover part 1406 are sealed by a cap when liquid is not being consumed, for example.

The cover parts 1404, 1406 also represent another example of a dosage device. In FIG. 14B, the cover parts 1404, 1406 are in a first state in which the cannabinoid-containing substance 1440 is held in the compartment 1432. In this first state, the surface 1412 defines a bottom surface of the compartment 1432, and the compartment inhibits the addition of the cannabinoid-containing substance 1440 to the liquid. However, the cover parts 1404, 1406 are movable relative to each other to provide a second state in which at least a portion of the cannabinoid-containing substance 1440 is added, or at least can be added, to liquid from the container body 1402. In the second state, the cover parts 1404, 1406 enable fluid communication between the compartment 1432 and the container body 1402. Specifically, in the example shown the cover part 1406 can be rotated relative to the cover part 1404 to align at least a portion the compartment 1432 with the opening 1414, and to release the cannabinoid-containing substance 1440 into the mixing chamber 1422. Depending on the form of the cannabinoid-containing substance 1440, the cannabinoid-containing substance can fall or flow into the mixing chamber 1422.

Once in the mixing chamber 1422, the cannabinoid-containing substance 1440 can mix with liquid from the container body 1402 in any of a number of ways. In some implementations, the mixing chamber 1422 is empty when the cannabinoid-containing substance 1440 is added. When a user drinks from the container 1400 using the orientation of the cover parts 1404, 1406 illustrated in FIG. 14B, the liquid flowing through the mixing chamber 1422 could mix with and/or dissolve the cannabinoid-containing substance 1440 to produce a cannabinoid-containing liquid. This cannabinoid-containing liquid could then be consumed by the user through the opening 1438. Some implementations include a filter, in the channel 1430 and/or one of the openings 1414, 1438 for example, to inhibit solid pieces or particles of the cannabinoid-containing substance from flowing into or through the channel 1430 and/or the opening 1438. For example, the cannabinoid-containing substance 1440 is in the form of a tablet in some embodiments, and the channel 1430 could include a filter disposed to filter fluid flow and block the tablet from moving into the channel 1430 or keep the table in the mixing chamber 1422 until it is dissolved or otherwise mixed with fluid from the container body 1402.

In some implementations, the mixing chamber 1422 is at least partially filled with liquid when the cannabinoid-containing substance 1440 is added. This liquid could be just a portion of the liquid in the container body 1402. The liquid could flow into the mixing chamber 1422 spontaneously during transport of the container 1400, for example. Alternatively, a user could shake, invert, and/or otherwise orient the container 1400 to cause liquid to flow into the mixing chamber 1422. When the cannabinoid-containing substance 1440 is added to the mixing chamber 1422, the cannabinoid-containing substance can mix with and/or dissolve in the liquid to produce a cannabinoid-containing liquid. The container 1400 may include one or more indicia or indicators such as an icon, to provide an operating instruction for a user to orient the container for fluid flow into the mixing chamber 1422 and add the cannabinoid-containing substance 1440 into the mixing chamber for mixing. The cannabinoid-containing liquid can then be consumed by a user when the cover parts 1404, 1406 are in the orientation shown in FIG. 14B.

In some implementations, the volume of the mixing chamber 1422 and the dosage of the cannabinoid-containing substance 1440 are selected or controlled by a user such that when the cannabinoid-containing substance is added to the mixing chamber, a cannabinoid-containing liquid having a predefined concentration is produced. Accordingly, the mixing chamber 1422 could provide a form of dosage control.

In some implementations, the cover part 1404 inhibits mixing of the cannabinoid-containing substance 1440 and a portion of the liquid that remains in the container body 1402. The cover part 1404 could also or instead inhibit contact of the cannabinoid-containing substance 1440 with the container body 1402, to prevent contamination of the container body. For example, the channel 1424 can inhibit the movement of the cannabinoid-containing substance 1440 from the mixing chamber 1422 into the container body 1402, at least when the container 1400 is held in the upright orientation illustrated in FIG. 14B. In some implementations, the container 1400 includes a filter, in the channel 1424 for example, to inhibit the movement of solid pieces or particles of the cannabinoid-containing substance 1440 from the mixing chamber 1422 to the container body 1402. The container 1400 could also or instead include a valve, in channel 1424 for example, to inhibit the flow of the cannabinoid-containing liquid from the mixing chamber 1422 to the container body 1402, but permit the flow of liquid from the container body to the mixing chamber. The valve could be a one-way valve, or a valve that only opens under sufficient pressure, such as when a user squeezes the container body 1402, for example. These implementations isolate the volume of liquid remaining in the container body 1402 from the mixing channel 1422, and inhibit that volume of liquid from being mixed in bulk with the cannabinoid-containing substance 1440. These implementations also inhibit the container body 1402 itself from being affected by the cannabinoid-containing substance 1440.

After a user drinks from the container 1400, there may be a volume of liquid that remains in the channel 1430. This volume of liquid could flow back into the mixing chamber 1422, or remain in the channel 1430. In some implementations, the mixing chamber 1422 is sized and shaped to maintain a pocket of air while a user is drinking from the container 1400. This pocket of air could accommodate any liquid that remains in the channel 1430 after the user drinks from the container 1400, and inhibit overfilling of the mixing chamber 1422 that could result in liquid flowing back into the container body 1402. In some implementations, the container 1400 includes a valve that enables the controlled addition of liquid into the mixing chamber 1422. For example, a switch or dial could be provided on the exterior of the container 1400 to control the flow of liquid through the opening 1426 and/or the channel 1424. This could enable a user to adjust the quantity and concentration of cannabinoid-containing liquid that is produced in the mixing chamber 1422.

The container 1400 provides one example of a mixing chamber. Other mixing chambers with different sizes, shapes and orientations are also contemplated. For example, some mixing chambers could be integrally formed with a container body. In some embodiments, a container, cover and/or dosage device could include more than one mixing chamber. Moreover, one or more mixing chambers could be implemented in containers with more than one cannabinoid-containing substance and/or compartment. Other features such as those disclosed by way of example elsewhere herein may be implemented in embodiments that are consistent with the example container 1400.

Several embodiments disclosed herein refer to emulsions, emulsification, emulsion systems, emulsification-based systems, and/or spray-drying or spray-dried substances. Such features are further described by way of example below.

Cannabinoids may be provided as compositions to form any of a large variety of cannabis infused products. To this end, cannabinoids present in cannabis plants may be extracted, concentrated, and provided in suitable compositions for use in such cannabis infused products, as further described herein.

Cannabinoid dry formulations suitable for use in cannabis infused products such as beverages, human or pet edibles, confectionaries, and the like, may be obtained by spray drying of cannabinoid emulsions, for example. Such products are expected to grow in popularity due to the existing and/or expected legalization of these product forms in Canada and other countries. As a result, attention has turned to how to prepare industrial scale quantities of these products to meet consumer demands. One approach is to provide a concentrated pre-mix formulation of the cannabis extract that could be easily shipped to a manufacturer. The manufacturer would then dilute the concentrated pre-mix formulation into different product bases to form a large variety of different beverages, including alcoholic and non-alcoholic beverages for example, human edibles such as chewing gums and mints or example, pet edibles such as pet food and pet chews for example, and/or confectionaries such as lozenges for example, that are ready for commercial sale and consumption.

A key challenge is to ensure that the cannabinoids are sufficiently solubilized in the concentrated pre-mix formulation all the way to the final cannabis infused products. Because cannabis formulations are typically highly lipophilic or “fat-loving” and have poor aqueous solubility or are essentially water insoluble, emulsification-based systems used for the solubilisation of concentrated pre-mix formulations have been described that are able to satisfy some or preferably all of the following objectives: (i) improved water solubility of the cannabinoids to maximize the consumable limits of the cannabis, within regulatory limits of 10 mg of cannabis per beverage package for Canada for example, (ii) storage stability over an expected shelf-life, of at least 6 months for example, (iii) transport stability over varying travel conditions including extreme temperatures, excessive agitation, and/or other travel or transport conditions for example, (iv) preferred or desired physical appearance, such as clear physical appearance for clear products or no discoloration for opaque products and/or no adverse effects such as ringing and/or creaming, for example, and (v) pleasant organoleptic properties, such as pleasing taste and/or smell for example.

Cannabinoid formulations comprising such emulsification-based systems, also referred to herein as emulsions or cannabinoid emulsions for example, generally include droplets of a carrier oil containing solubilized cannabinoids, the droplets being dispersed throughout a continuous aqueous phase. As such, these cannabinoid emulsions may notably be characterized by a particle size distribution (PSD) of the droplets in the cannabinoid emulsion. It has been shown that the PSD of such cannabinoid formulations may be correlated to the absorption of cannabinoids in an organism once the cannabinoid formulations is ingested.

However, cannabinoid emulsions exhibit a liquid or substantially liquid form, such as a liquid or a slurry for example, and as such may not be readily suitable for use in all cannabis infused products. For example, such emulsions may be well-suited for use in cannabis-infused beverages, but less so for use in cannabis-infused human or pet edibles for which admixing with a formulation exhibiting a substantially dry form such as a powder may be preferable. Powders may also be easier to transport and formulate and exhibit a longer shelf-life than emulsions, and may be useful, for example, in a dosage device for controlled addition to a liquid or container.

Spray drying is a conventional chemical process used to produce dry particulate solids such as dry powders, from a variety of liquid or substantially liquid materials. Spray drying processes for producing powders are well-known and disclosed, for example, in U.S. Pat. Nos. 5,976,574, 5,985,248, 6,001,336, 6,051,256, 6,077,543, and 6,423,344 and PCT Publications WO 96/32149, WO 99/16419, WO 01/00312, WO 01/85136 and in WO 02/09669. Dry powders obtained using such processes may also be re-hydrated. However, spray drying of cannabinoid emulsions using the processes described in these referenced documents may be detrimental to at least some of the properties of the re-hydrated formulations so obtained, such that at least some of the properties of the re-hydrated formulations obtained post-spray drying may be different from at least some of the properties of the original cannabinoid emulsions, including but not limited to the PSD of the droplets of carrier oil in which the cannabinoids are solubilized. This in turn may negatively impact the potential use of such re-hydrated formulations in cannabis-infused beverages, human or pet edibles and confectionaries, as well as other properties generally related to the reconstitution of cannabinoid emulsions and the absorption of the cannabinoids in the organism once ingested. Cannabinoid dry formulations that do not exhibit at least some of these shortcomings, and specifically cannabinoid dry formulations that are re-hydratable and that upon such re-hydration retain at least some of the properties of the original cannabinoid emulsions from which the cannabinoid dry formulation was obtained, are described by way of example herein at least below. Retained properties may include but are not limited to the PSD of the droplets of carrier oil in which the cannabinoids are solubilized.

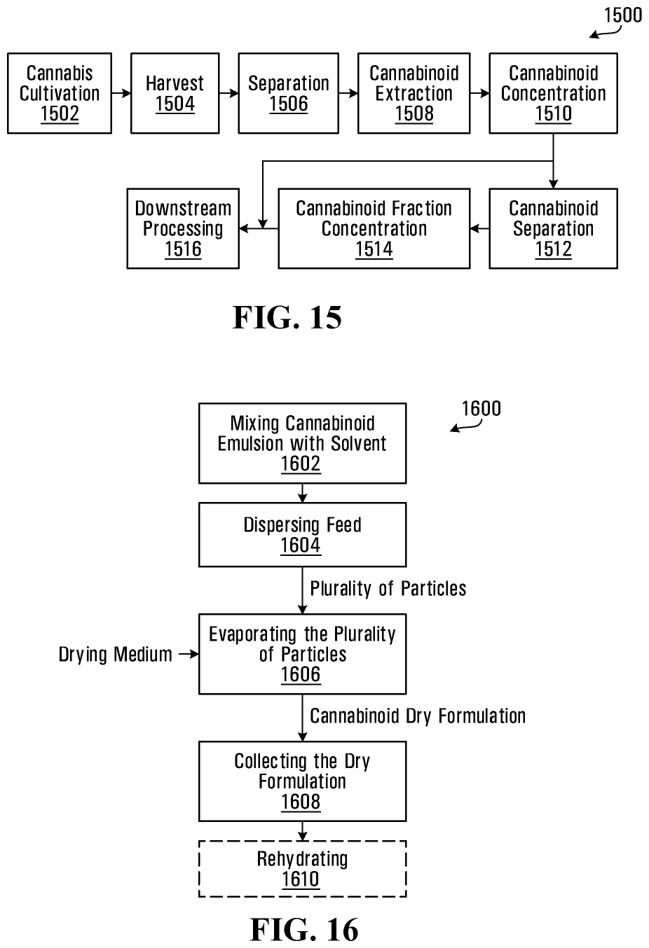

FIG. 15 shows a flow diagram illustrating a process 1500 for processing cannabis material to produce a cannabis composition to be used in one or more cannabis infused products, in accordance with one non-limiting embodiment. At step 1502, cannabis plants which contain cannabinoids are cultivated in a grow area and then are harvested at step 1504 to obtain cannabis plant material. Cannabis plant material is intended to include any material that originates from a cannabis plant, including cannabis flowers, trims and/or waste for example. Cannabis flowers could also be referred to as buds, and are typically harvested from mature cannabis plants. Trims includes the leaves of the cannabis plant that are separated from the flowers and stems. Trims could be harvested before the flowers, as the plants mature. Waste could include roots, stalks, stems and leaves that were not separated into trims, for example. The cannabis plant material obtained at the end of step 1504 is then subjected to a separation step 1506 that separates the cannabis plant material into distinct flower, trim and/or waste fractions. It will be readily appreciated that the separation step 1506 could alternatively be performed on cannabis plant material that is supplied from a cannabis producer. In one non-limiting example, at step 1506 the waste and the trims may be separated from the flowers.

The separated fraction obtained at the end of step 1506 is then subjected to a cannabinoid extraction step 1508. The cannabinoid extraction step 1508 may solubilize the cannabinoids present in the separated fraction, such as flowers, using an extraction solvent to form a first cannabinoid composition, namely a cannabinoid extract. As such, the cannabinoid extraction step 1508 is based at least in part on the solubility of the cannabinoids in an extraction solvent. Because cannabinoids are generally hydrophobic, one or more hydrophobic solvents in which the cannabinoids are soluble are used as extraction solvents. The cannabinoid extraction step 1508 includes processing or contacting the separated fraction with the extraction solvent, which separates the cannabinoids from the separated fraction and captures them in the form of an extract. Any material that is not in the extract and remains after the extraction is either treated as waste or subject to further processing. In some non-limiting examples, the extraction may be performed using any suitable (hydrophobic) solvent, such as but not limited to alcohol, hexane, propane, pentane, butane, acetone, and other hydrocarbons. In other non-limiting examples, the extraction solvent may be supercritical CO₂. Solvent-based extraction is one example of an extraction process. Mechanical extraction to separate trichomes, for example, may be used in other non-limiting embodiments. Other non-limiting embodiments could employ other types of extraction and/or multiple types of extraction, such as but not limited to liquid-liquid extraction, solid-phase extraction, solid-phase microextraction, Soxhlet extraction and fizzy extraction.

At step 1510, the cannabinoid extract obtained at the end of step 1508 is concentrated to increase the concentration of the extracted cannabinoids in the solvent to obtain a concentrated cannabinoid extract. In one non-limiting example, where a solvent extraction was used at step 1508, a purpose of step 1510 may be to eliminate at least a fraction of the solvent that was used at step 1508 to reduce the volume of the cannabinoid extract obtained at the end of step 1508 and therefore increase the concentration of the extracted cannabinoids in the solvent. In one non-limiting example, the concentration step 1510 may be performed using a rotary evaporator in which the solvent is removed from the extracted cannabinoid solution by evaporation. At step 1512, the cannabinoids present in the concentrated cannabinoid extract obtained at the end of step 1510 may then be separated to obtain distinct solubilized fractions each with a specific cannabinoid, for example one with THC, one with CBD, and so on. In one non-limiting example, the separation may be performed using high-performance liquid chromatography (HPLC) since distinct cannabinoids travel at different speeds through a suitable HPLC column (or stationary phase) such that they may each be eluted from the HPLC column at different times. At step 1514, the specific cannabinoid fractions obtained at the end of step 1512 may then be subjected to a further concentration step 1514 generally similar to step 1510 above, such as using a rotary evaporator to remove at least a fraction of the solvent by evaporation. The resulting concentrated specific cannabinoid fractions may then be subjected to further downstream processing at step 1516 to produce a variety of cannabis products, including cannabis infused products, as further described by way of example below and/or elsewhere herein. It will also be readily appreciated that, in some non-limiting embodiments, steps 1512 and 1514 may be omitted such that the concentrated extract obtained at the end of step 1510, which may contain a plurality of distinct cannabinoids, may be used directly as source material for the downstream processing at step 1516. Any other process for processing cannabis material may be suitable in other non-limiting embodiments.

The concentrated cannabinoid extract obtained at the end of step 1510 or the concentrated specific cannabinoid fractions obtained at the end of step 1514 are compositions comprising one or more (hydrophobic) cannabinoids solubilized in a (hydrophobic) solvent, the cannabinoids having poor water aqueous solubility. For the subsequent use in cannabis infused products, in one non-limiting embodiment, aqueous compositions comprising cannabinoids ought to be produced. Suitable examples include mixtures, suspensions or emulsions, preferably emulsions, even more preferably oil-in-water emulsions. In one non-limiting embodiment, the concentrated cannabinoid extract obtained at the end of steps 1510 or the concentrated specific cannabinoid fractions obtained at the end of step 1514 may be subjected to a variety of downstream processing steps 1516 so as to be provided as cannabinoid emulsions comprising a carrier oil or solvent in which one or more cannabinoids are solubilized, one or more emulsifiers and an aqueous solution such as water.

In one non-limiting example, the cannabinoid emulsion may exhibit water content of at least about 10 wt %, in some cases at least about 20 wt %, in some cases at least about 30 wt %, in some cases at least about 40 wt % and in some cases even more. The water content as used herein means the total amount of water present in the cannabinoid emulsion, whether added separately or as a solvent or carrier for other raw materials. In another non-limiting example, the cannabinoid emulsion may exhibit a water activity a_{w} of at least about 0.7, in some cases at least about 0.75, in some cases at least about 0.8, in some cases at least about 0.85, in some cases at least about 0.9, in some cases at least about 0.95 and in some cases even more.

The purpose of the carrier oil or solvent is to aid in solubilizing the hydrophobic cannabinoids in the emulsion. Non-limiting examples of suitable carrier oils or solvents include, but are not limited to, borage oil, coconut oil, cottonseed oil, soybean oil, safflower oil, sunflower oil, castor oil, corn oil, olive oil, palm oil, peanut oil, almond oil, sesame oil, rapeseed oil, peppermint oil, poppy seed oil, canola oil, palm kernel oil, hydrogenated soybean oil, hydrogenated vegetable oils, glyceryl esters of saturated fatty acids, glyceryl behenate, glyceryl distearate, glyceryl isostearate, glyceryl laurate, glyceryl monooleate, glyceryl, monolinoleate, glyceryl palmitate, glyceryl palmitostearate, glyceryl ricinoleate, glyceryl stearate, polyglyceryl 10-oleate, polyglyceryl 3-oleate, polyglyceryl 4-oleate, polyglyceryl 10-tetralinoleate, behenic acid, medium-chain triglycerides (e.g., caprylic/capric glycerides), ethanol, acetone, isopropanol, hydrocarbons or a combination thereof.

The purpose of the one or more emulsifiers is to act as surfactants and to reduce a surface tension at an interface between the carrier oil and the aqueous solution. The one or more emulsifiers may be ionic, non-ionic or a combination of both. In some non-limiting examples, the emulsifier may be a polysorbate, polyoxyethylene, polyoxypropylene block co-polymer, ethoxylated aliphatic alkyl alcohol, ethoxylated fatty alcohol, ethoxylated aliphatic alkyl acid, ethoxylated fatty acid, glyceryl monostearate, sorbitan fatty acid ester, capril caprylic macrogoglyceride, propylene glycol laurate, propylene glycol caprylate, glycerol monostrearate, polyglycerol oleate, lecithin-based emulsifier, tocopherol, polyoxyethylene or any combination therefore, and specifically polyoxyethylene monostearate (PEG 400 Monostearate), polyoxyethylene monooleate (PEG 400 Monoleate), polyoxyethylene sorbitan monolaurate (Tween® 20), polyoxyethylene sorbitan monolaurate (Tween® 21), polyoxyethylene sorbitan monopalmitate (Tween® 40), polyoxyethylene sorbitan monostearate (Tween® 60), polyoxyethylene sorbitan monostearate (Tween® 61), polyoxyethylene sorbitan tristearate (Tween® 65), polyoxyethylene sorbitan monooleate (Tween® 80), polyoxyethylene sorbitan monooleate (Tween 81), polyoxyethylene sorbitan trioleate (Tween® 85), polyoxyethylene-(15)-stearic acid (Pegosperse 1500MS), polyoxyethylene-(20)-stearyl alcohol (Brij 78), polyoxyethylene-(23)-lauryl alcohol (Brij 35), (Lutensol ON 60), PEG-40 hydrogenated castor oil (Cremophor/Kolliphor RH 40), PEG-35 castor oil (Cremophor EL), Solutol HS-15, sorbitan monopalmitate (Span 40), sorbitan monostearate (Span 60), sorbitan tristearate (Span 65), sorbitan monooleate (Span 80), sorbitan trioleate (Span 85), sunflower lecithin emulsifier, soybean lecithin emulsifier, linseed lecithin emulsifier, olive lecithin emulrapeseed lecithin emulsifier, egg lecithin emulsifier, corn lecithin emulsifier, peanut lecithin emulsifier, algal lecithin emulsifier, Vitamin E and Vitamin E derivatives (alpha, beta, gamma and delta-tocopherols), preferably d-alpha-tocopherol polyethyleneglycol 1000 succinate (Vitamin E TPGS), blend of isomers of alpha-tocopherol, beta-tocopherol, gamma-tocopherol and delta-tocopherol (Tocobiol®), polyoxyethylene (2) cetyl ether (Brij C2) and any combination thereof. Any other suitable non-ionic emulsifier may be used in other non-limiting examples.

In some non-limiting examples, the one or more emulsifiers may be present in an amount of from about 0.1 wt % to about 15 wt %, preferably from about 2 wt % to about 12 wt %, based on the total weight of the cannabinoid emulsion. In yet further non-limiting examples, the cannabinoid emulsion comprising the carrier oil in which one or more cannabinoids are solubilized, the one or more emulsifiers and the aqueous solution may operate to solubilize at least about 0.5 mg of cannabinoid in 1 mL of the aqueous solution, in some cases at least about 1 mg of cannabinoid in 1 mL of the aqueous solution, in some cases at least about 2 mg of cannabinoid in 1 mL of the aqueous solution, in some cases at least about 5 mg of cannabinoid in 1 mL of the aqueous solution and in some cases even more.

The cannabinoid emulsion may be characterized in a number of ways, for example using its particle size distribution (PSD). The term “particle size”, as used herein, refers to a volume based particle size measured, for example, by laser diffraction method. Laser diffraction measures particle size distribution by measuring the angular variation in intensity of light scattered as a laser beam passes through a dispersed particulate sample. Large particles scatter light at small angles relative to the laser beam and small particles scatter light at large angles. The angular scattering intensity data is then analyzed to calculate the size of the particles responsible for creating the scattering pattern, using the Mie theory of light scattering. The particle size is reported as a volume equivalent sphere diameter. Alternatively, the PSD can be measured by laser diffraction according to ISO 13320:2009 and ISO 9276-2:2014. The PSD of the emulsion is a parameter which contributes to solubilisation of cannabinoids, control of on-set and off-set of cannabinoids, specific turbidity and creaming stability of an emulsion.

It will be readily appreciated that the PSD of the cannabinoid emulsion refers to the PSD of the carrier oil droplets, as particles containing the solubilized cannabinoids, that are present in the aqueous solution. As such, in one non-limiting example, the cannabinoids may be encapsulated in the carrier oil droplets, the carrier oil droplets having a PSD of about 20 μm or less, in some cases about 10 μm or less, in some cases about 5 μm or less, in some cases about 2.5 μm or less, in some cases about 1 μm or less, in some cases about 750 nm or less, in some cases about 500 nm or less, in some cases about 250 nm or less, in some cases about 100 nm or less, in some cases about 50 nm or less, in some cases about 25 nm or less and in some cases even less.

In another non-limiting example, the cannabinoids may be encapsulated in the carrier oil droplets, the carrier oil droplets having a D₉₀ of about 20 μm or less, in some cases about 10 μm or less, in some cases about 5 μm or less, in some cases about 2.5 μm or less, in some cases about 1 μm or less, in some cases about 750 nm or less, in some cases about 500 nm or less, in some cases about 250 nm or less, in some cases about 100 nm or less, in some cases about 50 nm or less, in some cases about 25 nm or less and in some cases even less. The term “D₉₀” means the particle size of no more than 90% of the total amount of particles. For example, a D₉₀ of 100 nm or less means that no more than 90% of the total amount of particles may have a particle size of 100 nm or less.

In yet another non-limiting example, the cannabinoids may be encapsulated in the carrier oil droplets, the carrier oil droplets having a D₅₀ of about 20 μm or less, in some cases about 10 μm or less, in some cases about 5 μm or less, in some cases about 2.5 μm or less, in some cases about 1 μm or less, in some cases about 750 nm or less, in some cases about 500 nm or less, in some cases about 250 nm or less, in some cases about 100 nm or less, in some cases about 50 nm or less, in some cases about 25 nm or less and in some cases even less. The term “D₅₀” means the particle size of no more than 50% of the total amount of particles. For example, a D₅₀ of 100 nm or less means that no more than 50% of the total amount of particles may have a particle size of 100 nm or less.

The PSD of the cannabinoid emulsion may be controlled so as achieve a controlled onset and controlled offset of the cannabinoids once ingested, or a fast or delayed onset of the cannabinoids once ingested. In one non-limiting example, the cannabinoid emulsion may have a PSD < about 200 nm to impart the fast onset of the cannabinoids once ingested, in some cases a PSD ≤ about 100 nm, in some cases a PSD ≤ about 80 nm, in some cases a PSD ≤ about 70 nm, in some cases a PSD ≤ about 60 nm, in some cases a PSD about 50 nm, in some cases a PSD about 40 nm, in some cases a PSD ≤ about 30 nm, in some cases a PSD ≤ about 20 nm, in some cases a PSD ≤ about 10 nm and in some cases even less. Preferably, to impart the fast onset of the cannabinoids once ingested the cannabinoid emulsion has a PSD of from about 10 nm to about 80 nm, in some cases from about 10 nm to about 60 nm, in some cases from about 10 nm to about 40 nm, or any other value in-between. The fast onset may reflect the case where the tₘₐₓ of the cannabinoid in a subject having ingested a cannabis-infused edible or beverage containing the herein described cannabinoid emulsion with the PSD to impart the fast onset is significantly faster than with conventional cannabis-infused edibles or beverages. For example, a fast onset may be characterized as a tₘₐₓ of the cannabinoid in a subject having ingested the cannabis-infused edibles or beverages containing the herein described cannabinoid emulsion with the PSD to impart the fast onset within the range of from about 15 minutes to about 1 hour 45 minutes, or from about 15 minutes to about 1 hour 30 minutes, or from about 15 minutes to about 1 hour 15 minutes, or from about 15 minutes to about 1 hour, or from about 15 minutes to about 45 minutes, or from about 15 minutes to about 30 minutes, including any values therein.

In one non-limiting example, the controlled offset may be achieved by means of a distinct emulsion comprising at least one of an antidote, attenuator or modulator that modulates the absorption of the cannabinoids contained in the cannabinoid emulsion once ingested. That is, much like the cannabinoid emulsion, the at least one antidote, attenuator or moderator is contained within droplets of carrier oil or solvent present in the aqueous solution. The antidote, attenuator or modulator emulsion has a PSD that is greater than the PSD of the cannabinoid emulsion such that the PSD of the antidote, attenuator or modulator emulsion imparts the delayed onset of the antidote, attenuator or moderator (i.e., the controlled offset of the cannabinoids). In some non-limiting examples, the antidote, attenuator or modulator emulsion may have a PSD of > about 200 nm to impart the delayed onset of the antidote, attenuator or modulator once ingested, in some cases a PSD ≥ about 300 nm, in some cases a PSD about 400 nm, in some cases a PSD ≥ about 500 nm, in some cases a PSD ≥ about 600 nm, in some cases a PSD ≥ about 700 nm, in some cases a PSD ≥ about 800 nm, in some cases a PSD ≥ about 900 nm, in some cases a PSD ≥ about 1000 nm and in some cases even more. It will be readily appreciated that when both the cannabinoid emulsion and the antidote, attenuator or modulator emulsion are ingested simultaneously, there is a differential absorption rate of the cannabinoid emulsion and the antidote, attenuator or modulator emulsion, with the cannabinoid emulsion absorbing faster once ingested than the antidote, attenuator or modulator emulsion, thus resulting in a faster onset associated with the cannabinoids relative to the onset of the antidote, attenuator or modulator of the cannabinoids. The controlled offset may reflect the case where the tₘₐₓ of the cannabinoid in a subject having ingested a cannabis-infused edible or beverage comprising the herein described antidote, attenuator or modulator emulsion decreases by at least about 50% (e.g., 50%, 55%, 60%, 65%, 70%, 75%, 80%, 85%, 90%, 95% or any value therein) in less than about 3 hours from the time of tₘₐₓ, such as for example in less than about 2 hours 30 minutes from the time of tₘₐₓ, or in less than about 2 hours 15 minutes from the time of tₘₐₓ, in less than about 2 hours from the time of tₘₐₓ, or in less than about 1 hour 45 minutes from the time of tₘₐₓ, or in less than about 1 hour 30 minutes from the time of tₘₐₓ, or in less than about 1 hour 15 minutes from the time of tₘₐₓ, or in less than about 1 hour from the time of tₘₐₓ, or in less than about 45 minutes from the time of tₘₐₓ, or in less than about 30 minutes from the time of tₘₐₓ.

In one non-limiting example, the at least one antidote, attenuator or modulator may include one or more compound selected from cannabidiol (CBD), acorus calamus or extracts thereof, black pepper or extracts thereof, citrus or extracts thereof, pine nuts or extracts thereof, pistachio nuts or extracts thereof, fruits of Pistacia terebinthus or extracts thereof, piperine, or terpenes, such as β-caryophyllene, limonene, myrcene, or α-pinene.

In another non-limiting example, the cannabinoid emulsion may have a PSD of > about 200 nm to impart the delayed onset of the cannabinoids once ingested, in some cases a PSD ≥ about 300 nm, in some cases a PSD ≥ about 400 nm, in some cases a PSD ≥ about 500 nm, in some cases a PSD ≥ about 600 nm, in some cases a PSD ≥ about 700 nm, in some cases a PSD ≥ about 800 nm, in some cases a PSD ≥ about 900 nm, in some cases a PSD ≥ about 1000 nm and in some cases even more. It will be readily appreciated that the cannabinoid emulsion having the PSD to impart the fast onset of the cannabinoids once ingested as described above will exhibit a faster absorption rate once ingested that the cannabinoid emulsion having the PSD to impart the delayed onset of the cannabinoids once ingested.

In other non-limiting examples, either one of the cannabinoid emulsion or the antidote, attenuator or modulator emulsion may further comprise at least one agent that further modulates the absorption of the cannabinoids contained in the cannabinoid emulsion once ingested, such as but not limited to a mucolytic, an efflux blocker, or any combinations thereof.

In yet further non-limiting examples, the cannabinoid emulsion may further comprise a liquid carrier, such as for example, water, preferably USP water. The water may be added as an ingredient on its own right or it may be present as a carrier in other common raw materials. In other non-limiting examples, the cannabinoid emulsion may include one or more other components such as, for example, a co-solvent, a preservative, or a buffering agent.

With further reference to FIG. 16, a non-limiting embodiment of a spray-drying process 1600 for drying, or substantially drying, a cannabinoid emulsion such as the emulsion described above is provided. At step 1602, the cannabinoid emulsion is mixed with a solvent for further processing, the mixture of the cannabinoid emulsion with the solvent being referred to as feed. The solvent may be a solubilized sugar carrier and the sugar carrier may be any suitable sugar, such as but not limited to polysaccharides such as maltodextrin and soy soluble polysaccharides, cyclodextrin, mannitol, gum arabic, starches such as corn starch, modified starches such as octenyl succinate modified starches, modified cellulose such as methyl cellulose, hydroxypropyl cellulose, methyl hydroxypropyl cellulose, and carboxymethylcellulose, certain types of pectin such as beet pectin, corn fiber gum and the like.

In one non-limiting example, a concentration of the sugar carrier in the solvent (in w/w) may be about 0.1%, in some cases about 0.5%, in some cases about 1%, in some cases about 1.5%, in some cases about 2%, in some cases about 2.5%, in some cases about 5%, in some cases about 10%, in some cases about 15%, in some cases about 20%, in some cases about 25%, in some cases about 50% and in some cases even more.

In another non-limiting example, a ratio of solvent to cannabinoid emulsion (in v/v) in the feed may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

In yet another non-limiting example, the feed may have a water content that is at least about 10 wt %, in some cases at least about 20 wt %, in some cases at least about 30 wt %, in some cases at least about 40 wt % water and in some cases even more. In yet a further non-limiting example, the feed may have a water activity a_{w} of at least about 0.7, in some cases at least about 0.75, in some cases at least about 0.8, in some cases at least about 0.85, in some cases at least about 0.9, in some cases at least about 0.95 and in some cases even more. Because the feed may generally be considered as being in a liquid or substantially liquid phase, the spray-drying process 1600 will effect a transition from a liquid phase to a dry (or substantially dry) phase of the feed, the dry (or substantially dry) phase of the feed comprising a plurality of dried feed particles, as further described below.

In other non-limiting embodiments, additives may also be added to the feed for conformational stability during spray drying and for improving the dispersibility of the eventual cannabinoid dry formulation. These additives include but are not limited to hydrophobic amino acids such as tryptophan, tyrosine, leucine, phenylalanine, and the likes. pH adjusters or buffers such as but not limited to organic salts prepared from organic acids and bases (e.g., sodium citrate, sodium ascorbate) and the like may also be added to the feed in other non-limiting embodiments.

At step 1604, the feed is dispersed by running the feed through an atomizer, dispersion referring to the process of atomizing the feed (comprising the cannabinoid emulsion mixed with the solvent) into a plurality of particles (or droplets). As such, the purpose of step 1604 is to increase a surface area of the feed via the formation of the plurality of particles. Due to atomization there is an increase in the surface energy of the liquid, the magnitude of which is directly proportional to the increase of the surface area of the feed. The source of this energy increase depends on the type of atomizer used. The atomizer used at step 1604 may be any suitably atomizer, such as but not limited to a pressure nozzle, a two-fluid nozzle, an ultrasonic nozzle, a rotary disc atomizer and the likes, the selection of a particular type of atomizer being reliant at least upon the nature and the amount of the feed being provided at step 1602 as well as the desired characteristics of the eventual plurality of dried feed particles, which constitute the dry cannabinoid formulation.

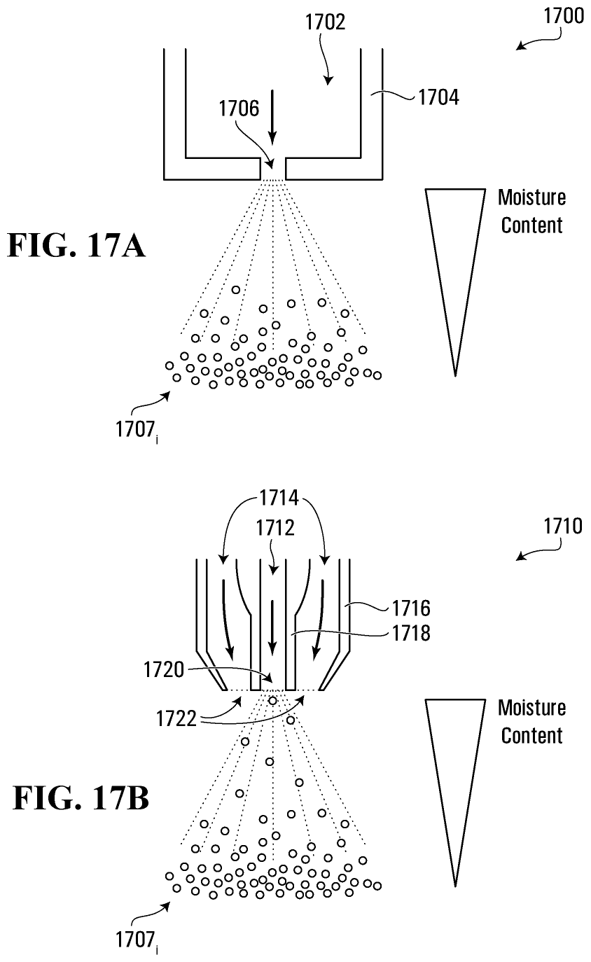

With further reference to FIG. 17A, a non-limiting example of a pressure nozzle 1700 that may be used as atomizer at step 1504 is provided. The pressure nozzle comprises an outer circumferential wall 1704 defining an inner compartment 1702 through which the feed is provided and an opening 1706 through which the feed may exit the compartment 1702 and be atomized in the plurality of particles 1707ᵢ. Generally, the higher the pressure of the feed within the compartment 1702, the higher the flow of the feed through the opening 1706 and the smaller the size of the plurality of particles 1707ᵢ at the exit of the opening 1706. Smaller plurality of particles 1707ᵢ at the exit of the opening 1706 will ultimately result in smaller dried particles after the evaporating step 1606, as further described below. Therefore, the pressure nozzle 1700 atomizes the feed into the plurality of particles 1707ᵢ by relying solely on the kinetic energy of the feed within the compartment 1702. While the pressure nozzle 1700 shown in FIG. 17A only exhibits a single opening 1706, a plurality of openings may be also present and any other suitable configuration of the pressure nozzle 1700 (e.g., geometrical configuration of the opening 1706, presence of a surface impingement, pressure swirl, compound nozzles, etc.) may be suitable in other non-limiting examples.

With further reference to FIG. 17B, a non-limiting example of a two-fluid nozzle 1710 that may be used as atomizer at step 1604 is also provided. The two-fluid nozzle 1710 comprises an outer circumferential wall 1716 and an inner circumferential wall 1718, the outer circumferential wall 1716 and the inner circumferential wall 1718 defining: (i) a first inner compartment 1712 through which the feed is provided; (ii) a second inner compartment 1714 through which a fluid, or atomization fluid, is provided; (iii) an opening 1720 through which the feed may exit the compartment 1712 and be atomized in the plurality of particles 1707ᵢ, and (iv) an opening 1722 through which the atomization fluid may exit the compartment 1714 and contact the feed as it exits the compartment 1712. The pressure nozzle 1710 atomizes the feed into the plurality of particles 1707ᵢ by relying on the interaction between the flowing feed and the atomization fluid at the exit of the two-fluid nozzle 1710. Much like the pressure nozzle 1700 of FIG. 17A, the higher the pressure of the feed within the compartment 1712, the higher the flow rate of feed through the opening 1720 and the smaller the size of the plurality of particles 1707ᵢ at the exit of the opening 1706. Similarly, the higher the pressure of the fluid within the compartment 1714, the smaller the size of the plurality of particles 1707ᵢ at the exit of the opening 1706. Again, smaller plurality of particles 1707ᵢ at the exit of the opening 1720 will ultimately result in smaller dried particles after the evaporating step 1606, as further described below.

In this non-limiting example, the atomization fluid may be a gas such as compressed air, nitrogen or any other suitable gas and the gas is filtered or otherwise cleaned to remove particulates and other contaminants. The gas is pressurized for delivery through the opening 1722, for example to a pressure of at least about 1 psig, in some cases at least about 2.5 psig, in some cases at least about 5 psig, in some cases at least about 10 psig, in some cases at least about 15 psig and in some cases even more. While the two-fluid nozzle 1700 of FIG. 17B has a configuration through which the feed and the atomization fluid are mixed externally to the two-fluid nozzle 1700, any other suitable configuration is possible in other non-limiting examples (e.g., with internal mixing of the feed and fluid, etc.).

It will be readily appreciated that, as the plurality of particles 1707ᵢ are formed at the exit of the atomizer, the plurality of particles 1707ᵢ may not be dried or substantially dried, that is, the plurality of particles 1707ᵢ may still retain a totality, a majority or a substantiality of the water content and water activity of the feed comprising the cannabinoid emulsion mixed with the solvent. At the exit of the atomizer, the water content and the water activity of the plurality of particles 1707₁ may therefore be substantially identical to that of the feed being fed to the atomizer at step 1604. The drying of the plurality of particles 1707ᵢ is performed as the plurality of particles 1707ᵢ travel away from the exit of atomizer at step 1606, notably as the plurality of particles 1707ᵢ come into contact with a drying medium. The contact of the drying medium with the plurality of particles 1707ᵢ will cause evaporation of the water present in the plurality of droplets 1707ᵢ, as further described below.

In one non-limiting embodiment, the drying medium may be air, an inert gas such as nitrogen or any other suitable gas that has been filtered or otherwise treated to remove particulates and other contaminants. The drying medium is flowed through the plurality of particles 1707ᵢ as they travel away from the atomizer up to a location where the particles will be collected, the flowing of the drying medium being performed using conventional blowers or compressors to move the drying medium from an inlet to an outlet. In some non-limiting examples, the flow rate of the drying medium via the inlet may be at least about 10 mL/hour, in some cases at least about 25 mL/hour, in some cases at least about 50 mL/hour, in some cases at least about 100 mL/hour, in some cases at least about 150 mL/hour, in some cases at least about 200 mL/hour, in some cases at least about 250 mL/hour, in some cases at least about 500 mL/hour, in some cases at least about 1000 mL/hour, in some cases at least about 2000 mL/hour and in some cases even more.

To cause evaporation of the water present in the plurality of droplets 1707ᵢ, the drying medium is heated and as such both the drying medium inlet and outlet temperatures should be controlled in order to control the residual water content and water activity at the end of the spray drying process. That is, in some non-limiting examples, a temperature of the drying medium at the inlet may be at least about 80° C., in some cases at least about 100° C., in some cases at least about 120° C., in some cases at least about 140° C., in some cases at least about 160° C., in some cases at least about 180° C. and in some cases even more. In other non-limiting examples, a temperature of the drying medium at the outlet may be at least about 50° C., in some cases at least about 60° C., in some cases at least about 70° C., in some cases at least about 80° C., in some cases at least about 90° C., in some cases at least about 100° C. and in some cases even more. It will be readily appreciated that the temperature of the drying medium at the outlet may be a function of at least the temperature of the drying medium at the inlet and the heat load imposed by the drying of the plurality of particles 1707ᵢ (the heat load being itself a function of the temperature of the feed, the quantity of water to be evaporated in the feed and the like).

In one non-limiting embodiment, the drying medium may be flowed in substantially the same direction as the direction of travel of the plurality of particles 1707ᵢ as they exit the atomizer. That is, in this embodiment, the inlet is positioned in a vicinity of the atomizer and the plurality of particles 1707ᵢ are contacted with a drying medium exhibiting a generally decreasing temperature profile as the plurality of particles 1707ᵢ travel away from the atomizer. This results in vaporization, specifically evaporation, of the water present in the plurality of particles 1707ᵢ, notably via evaporation of a saturated vapour film that is formed at a surface of the plurality of particles 1707ᵢ, thereby forming the plurality of dried feed particles, which constitute the dry cannabinoid formulation. In another non-limiting embodiment, the drying medium may be flowed in a direction substantially opposite to the direction of travel of the plurality of particles 1707ᵢ as they exit the atomizer. That is, in this embodiment, the plurality of particles 1707ᵢ are contacted with a drying medium exhibiting a generally increasing temperature profile as the plurality of particles 1707ᵢ travel away from the atomizer. As the water evaporates from the plurality of particles 1707ᵢ at step 1606, that is as the water content and the water activity of the plurality of particles 1707ᵢ decrease, the solubilized sugar carrier forms a sugar matrix around the droplets of carrier oil containing the solubilized cannabinoids that were originally present in the cannabinoid emulsion.

The chamber in which the plurality of particles 1707ᵢ are contacted with the drying medium may be configured such that a residence time of the plurality of particles 1707ᵢ within the chamber ensures drying of the plurality of particles 1707ᵢ to a prescribed water content or water activity, while also ensuring that the plurality of particles 1707ᵢ are collected before the temperature of the plurality of particles 1707ᵢ rises to levels that could be damaging to either one of the sugar matrix formed during the drying process or the droplets of carrier oil. For the purpose of this specification the residence time may be defined as the time needed for the plurality of particles 1707ᵢ to reach the prescribed water content and/or water activity. In one non-limiting example, the plurality of particles 1707ᵢ are effectively dried (i.e., the drying has resulted in the plurality of dried feed particles) when the prescribed water content and/or water activity have been reached.

It will be readily appreciated that a drying rate of the plurality of particles 1707ᵢ as they travel away from the atomizer may be dependent upon a plurality of parameters, such as but not limited to: (i) the overall surface area the plurality of particles 1707ᵢ; (ii) the temperature of the plurality of particles 1707ᵢ as they exit the atomizer; (iii) the temperature of the drying medium at the inlet and the outlet; (iv) the direction of flow of the drying medium relative to that of the plurality of particles 1707ᵢ as they travel away from the atomizer; (y) the humidity of the drying medium; and (vi) the drying medium flow rate through the plurality of particles 1707ᵢ.

At step 1608, the plurality of dried feed particles so obtained may be collected in a collection device to obtain the cannabinoid dry formulation. In one non-limiting example, the collection device may be a cyclone separator however conventional separation operations may also be used for example using a filter medium such as a membrane medium (bag filter), a sintered metal fiber filter and the likes. The cannabinoid dry formulation may be subsequently packaged for further use, as further described below. Optional, and possibly later, rehydration is shown at 1610.

The cannabinoid dry formulation, which consists in the plurality of dried feed particles collected at step 1608, may be characterized in a number of ways, including but not limited to by a PSD of the plurality of dried feed particles constituting the cannabinoid dry formulation, a surface area of the plurality of dried feed particles, a rugosity of the plurality of dried feed particles, a water content and a water activity. In one non-limiting example, the PSD of the plurality of dried feed particles may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less. In another non-limiting example, the D_{go} of the plurality of dried feed particles may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less. In yet another non-limiting example, the D₅₀ of the plurality of dried feed particles may be about 100 μm or less, in some cases about 50 μm or less, in some cases about 25 μm or less, in some cases about 10 μm or less, in some cases about 5 μm or less, in some cases about 2 μm or less, in some cases about 1 μm or less and in some cases even less. In yet another non-limiting example, the surface area of the plurality of dried particles per unit volume of feed may be at least about 0.5 m² per 100 mL of feed, in some cases at least about 1 m² per 100 mL of feed, in some cases at least about 2 m² per 100 mL of feed, in some cases at least about 3 m² per 100 mL of feed, in some cases at least about 4 m² per 100 mL of feed, in some cases at least about 5 m² per 100 mL of feed, in some cases at least about 10 m² per 100 mL of feed, in some cases at least about 15 m² per 100 mL of feed, in some cases at least about 20 m² per 100 mL of feed, in some cases at least about 25 m² per 100 mL of feed, in some cases at least about 50 m² per 100 mL of feed, in some cases at least about 100 m² per 100 mL of feed, in some cases at least about 150 m² per 100 mL of feed, in some cases at least about 200 m² per 100 mL of feed and in some cases even more.

As described above, it will be readily appreciated that the PSD of the plurality of dried feed particles may be dependent upon a number of parameters, including but not limited to: (i) the atomizer type; (ii) the operational parameters specific to the particular atomizer selected (e.g., feed pressure and flow, atomizing fluid pressure and flow, nozzle dimensions, opening size and numbers in the atomizer, atomizing fluid, etc.); (iii) the parameters of the evaporating step (e.g., drying medium type, drying medium temperature, drying medium humidity, drying medium flow direction, etc.); (iv) the mechanical properties of the feed (e.g., viscosity, etc.); and (v) the composition of the feed (e.g., solubilized sugar carrier concentration, type, etc.). The surface area of the plurality of dried feed particles may itself be dependent upon the PSD of the plurality of dried feed particles and the volume of feed subjected to dispersion at step 1604. The larger the volume of feed being provided, the larger the surface area of the plurality of dried feed particles. Similarly, the smaller the PSD, the larger the surface area of the plurality of dried feed particles.

In other non-limiting examples, the cannabinoid dry formulation may also be characterized by a rugosity of the plurality of dried particles, rugosity being a measure of surface convolution being defined as the ratio between the actual surface of a particle and the geometrical surface of the particle (assuming for example that the particle in spherical), with a higher number indicating a higher degree of surface irregularity. Specifically, the cannabinoid dry formulation may have a rugosity of at least about 1.3, in some cases at least about 1.4, in some cases at least about 1.5, in some cases at least about 1.6, in some cases at least about 1.7, in some cases at least about 1.8, in some cases at least about 1.9, in some cases at least about 2.0 and in some cases even more. It will be readily appreciated that an increase in the rugosity of the cannabinoid dry formulation may be correlated with a decrease in the cohesiveness and surface interactions between adjacent particles of the plurality of dried feed particles. As such, an increase in the rugosity of the cannabinoid dry formulation may be correlated with an increase in the dispersibility of the cannabinoid dry formulation upon re-hydration, as further described below. It will be further appreciated that the rugosity of the cannabinoid dried formulation may be dependent upon a variety of parameters, such as but not limited to: (i) the atomizer type; (ii) the operational parameters specific to the particular atomizer selected (e.g., feed pressure and flow, atomizing fluid pressure and flow, nozzle dimensions, opening size and numbers in the atomizer, atomizing fluid, etc.); (iii) the drying rate of the plurality of particles 1707ᵢ as they travel away from the atomizer; and (iv) the composition of the feed.

The term “dry” is understood to mean that the cannabinoid dry formulation has a water content and a water activity such that the cannabinoid dry formulation is physically and chemically stable in storage at room temperature. In some non-limiting examples, the water content of the cannabinoid dry formulation may be no more than about 10 wt %, in some cases no more than about 5 wt %, in some cases no more than about 3 wt % and in some cases even less. In yet other non-limiting examples, the water activity a_{w} of the plurality of dried feed particles may be no more than about 0.7, in some cases no more than about 0.65, in some cases no more than about 0.6, in some cases no more than about 0.55, in some cases no more than about 0.5, and in some cases even less.

In one non-limiting example, the cannabinoid dry formulation may have a shelf-life (i.e., a storage length during which the cannabinoid dry formulation does not become unfit for use and/or re-hydration) that is at least about 1 month, in some cases at least about 2 months, in some cases at least about 3 months, in some cases at least about 6 months, in some cases at least about 1 year, in some cases at least about 2 years and in some cases even more.

Much like in the cannabinoid emulsion, the carrier oil droplets (in which the cannabinoids are solubilized) present in the cannabinoid dry formulation may also be characterized by a PSD. In one non-limiting example, the carrier oil droplets present in the plurality of dried feed particles may exhibit a PSD that is substantially identical to, or the same as, that of the carrier oil droplets present in the cannabinoid emulsion, that is the PSD of the carrier oil droplets present in the plurality of dried feed particles does not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less compared to the PSD of the carrier oil droplets present in the cannabinoid emulsion that is subjected to the spray drying process described above.

In another non-limiting example, the carrier oil droplets present in the plurality of dried feed particles may exhibit a D₉₀ that is substantially identical to that of the carrier oil droplets present in the cannabinoid emulsion, that is the D₉₀ of the carrier oil droplets present in the plurality of dried feed particles does not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less compared to the D₉₀ of the carrier oil droplets present in the cannabinoid emulsion that is subjected to the spray drying process described above.

In yet another non-limiting example, the carrier oil droplets present in the plurality of dried feed particles may exhibit a D₅₀ that is substantially identical to that of the carrier oil droplets present in the cannabinoid emulsion, that is the D₅₀ of the carrier oil droplets present in the plurality of dried feed particles does not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less compared to the D₅₀ of the carrier oil droplets present in the cannabinoid emulsion that is subjected to the spray drying process described above.

In another non-limiting example, the PSD of the carrier oil droplets present in the plurality of dried feed particles, the D₉₀ of the carrier oil droplets present in the plurality of dried feed particles and/or D₅₀ of the carrier oil droplets present in the plurality of dried feed particles remain substantially similar or the same after a storage period of at least about 1 month at 40° C., in some cases at least about 2 months at 40° C., in some cases at least about 6 months at 40° C., in some cases at least about 1 year at 40° C. and in some cases even more. That is, the PSD of the carrier oil droplets present in the plurality of dried feed particles, the D₉₀ of the carrier oil droplets present in the plurality of dried feed particles and/or D₅₀ of the carrier oil droplets present in the plurality of dried feed particles do not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less over the time periods described above.

In one non-limiting embodiment, the cannabinoid dry formulation comprises an effective amount of cannabinoids for producing physiological effects associated with a feeling of physical and/or emotional satisfaction once formulated into the cannabis infused products (e.g., beverages, human or pet edibles, confectionaries). In another non-limiting embodiment, the cannabinoid dry formulation comprises an effective amount of the cannabinoid for treating or alleviating a disease or condition once formulated into the cannabis infused products (e.g., beverages, human or pet edibles, confectionaries). Preferably, the cannabinoid dry formulation comprises the cannabinoid present in an amount of from about 1 mg/g of formulation to about 50 mg/g of formulation, in some cases from about 4 mg/g of formulation to about 40 mg/g of formulation, or in some cases from about 10 mg/g of formulation to about 25 mg/g of formulation. Cannabinoids provided at such an amount in the cannabinoid dry formulation of the present disclosure can be particularly effective in delivering the desired physiological effects and/or treating or alleviating a disease or condition once formulated into the cannabis infused products. Such concentration of cannabinoid in the cannabinoid dry formulation may also be effective in delivering a desired onset of action once formulated into the cannabis infused products.

In another non-limiting embodiment, the types of cannabinoids and/or the levels of the cannabinoids incorporated into the cannabinoid dry formulation of the present disclosure provide substantially no psychoactive effect or no psychoactive effect. In other words, the types of cannabinoids and/or the levels of the cannabinoids used in the present cannabinoid dry formulation do not substantially or do not affect mood, perception, consciousness, cognition or behavior of a subject, as a result of changes in the normal functioning of the nervous system.

In another embodiment, it is desirable that various cannabinoids can be used in combination to achieve the desired effect. Suitable combinations of the cannabinoid which can be used in the present disclosure include a combination of THC and CBD. Certain specific ratios of cannabinoids may be useful to produce the feeling of physical and/or emotional satisfaction and/or may be useful in the treatment or management of specific diseases or conditions.

In some non-limiting examples, the (w/w) ratio of the THC to the CBD is between about 1:1000 and about 1000:1. Preferably, the (w/w) ratio of THC to CBD in the cannabinoid dry formulation may be about 1:1000, about 1:900, about 1:800, about 1:700, about 1:600, about 1:500, about 1:400, about 1:300, about 1:250, about 1:200, about 1:150, about 1:100, about 1:90, about 1:80, about 1:70, about 1:60, about 1:50, about 1:45, about 1:40, about 1:35, about 1:30, about 1:29, about 1:28, about 1:27, about 1:26, about 1:25, about 1:24, about 1:23, about 1:22, about 1:21, about 1:20, about 1:19, about 1:18, about 1:17, about 1:16, about 1:15, about 1:14, about 1:13, about 1:12, about 1:11, about 1:10, about 1:9, about 1:8, about 1:7, about 1:6, about 1:5, about 1:4.5, about 1:4, about 1:3.5, about 1:3, about 1:2.9, about 1:2.8, about 1:2.7, about 1:2.6, about 1:2.5, about 1:2.4, about 1:2.3, about 1:2.2, about 1:2.1, about 1:2, about 1:1.9, about 1:1.8, about 1:1.7, about 1:1.6, about 1:1.5, about 1:1.4, about 1:1.3, about 1:1.2, about 1:1.1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.1:1, about 2.2:1, about 2.3:1, about 2.4:1, about 2.5:1, about 2.6:1, about 2.7:1, about 2.8:1, about 2.9:1, about 3:1, about 3.5:1, about 4:1, about 4.5:1, about 5:1, about 6:1, about 7:1, about 8:1, about 9:1, about 10:1, about 11:1, about 12:1, about 13:1, about 14:1, about 15:1, about 16:1, about 17:1, about 18:1, about 19:1, about 20:1, about 21:1, about 22:1, about 23:1, about 24:1, about 25:1, about 26:1, about 27:1, about 28:1, about 29:1, about 30:1, about 35:1, about 40:1, about 45:1, about 50:1, about 60:1, about 70:1, about 80:1, about 90:1, about 100:1, about 150:1, about 200:1, about 250:1, about 300:1, about 400:1, about 500:1, about 600:1, about 700:1, about 800:1, about 900:1.

The cannabinoid dry formulation described above may be suitable for use in cannabis infused products (e.g., beverages, human or pet edibles, confectionaries, etc.). That is, the cannabinoid dry formulation may be added to a variety of beverages, human or pet edibles, confectionaries and the likes, as further described below.

In some non-limiting examples, the cannabinoid dry formulation may be used “as-is” in cannabis infused products, in which cases the cannabinoid dry formulation may be provided directly in its powder form for addition/mixing in the cannabis infused product. It will be readily appreciated that in the instances in which the cannabis infused product is a beverage (e.g., an aqueous solution), the addition/mixing to the beverage will rehydrate the cannabinoid dry formulation to form a rehydrated cannabinoid formulation.

In one non-limiting embodiment, the cannabinoid dry formulation may be rehydrated in an aqueous solution and the carrier oil droplets present in the rehydrated cannabinoid formulation may exhibit a PSD that is substantially identical to, or the same as, that of the carrier oil droplets present in the cannabinoid emulsion that is subjected to the spray-drying process described above to produce the cannabinoid dry formulation, that is the PSD of the carrier oil droplets present in the rehydrated cannabinoid formulation does not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less compared to the PSD of the carrier oil droplets present in the cannabinoid emulsion that is subjected to the spray drying process described above.

In another non-limiting example, the carrier oil droplets present in the rehydrated cannabinoid formulation may exhibit a D₉₀ that is substantially identical to that of the carrier oil droplets present in the cannabinoid emulsion that is subjected to the spray-drying process described above to produce the cannabinoid dry formulation, that is the D₉₀ of the carrier oil droplets present in the rehydrated cannabinoid formulation does not change by more than about 200%, in some cases by more than about 100%, in some cases by more than about 50%, in some cases by more than about 25%, in some cases more than about 20%, in some cases more than about 15%, in some cases by more than about 10%, in some cases by more than about 5%, in some cases by more than about 2%, in some cases by more than about 1% and in some cases even less compared to the D₉₀ of the carrier oil droplets present in the cannabinoid emulsion that is subjected to the spray drying process described above.

In yet another non-limiting example, the carrier oil droplets present in the rehydrated cannabinoid formulation may exhibit a D₅₀ that is substantially identical to that of the carrier oil droplets present in the cannabinoid emulsion that is subjected to the spray-drying process described above to produce the cannabinoid dry formulation, that is the D₅₀ of the carrier oil droplets present in the rehydrated cannabinoid formulation does not change by more than about 200%, in some cases by more than about 100%, in some cases by more than about 50%, in some cases by more than about 25%, in some cases more than about 20%, in some cases more than about 15%, in some cases more than about 10%, in some cases by more than about 5%, in some cases by more than about 2%, in some cases by more than about 1% and in some cases even less compared to the D₅₀ of the carrier oil droplets present in the cannabinoid emulsion that is subjected to the spray drying process described above.

In yet another non-limiting example, the tₘₐₓ of the cannabinoids in a subject having ingested the herein described rehydrated cannabinoid formulation is substantially identical to that of the cannabinoids in a subject having ingested the herein described cannabinoid emulsion that is subjected to the spray-drying process described above to produce the cannabinoid dry formulation that is rehydrated, that is tₘₐₓ of the cannabinoids in a subject having ingested the herein described rehydrated cannabinoid formulation does not change by more than about 25%, in some cases more than about 20%, in some cases more than about 15%, in some cases by more than about 10%, in some cases by more than about 5%, in some cases by more than about 2%, in some cases by more than about 1% and in some cases even less compared to the tₘₐₓ of the cannabinoids in a subject having ingested the herein described cannabinoid emulsion that is subjected to the spray-drying process described above to produce the cannabinoid dry formulation that is rehydrated.

The rehydrated formulation exhibits a PSD that is substantially identical to, or the same as, that of the carrier oil droplets present in the cannabinoid emulsion as described above when mixed with aqueous solutions exhibiting a variety of pH and salinity. That is, in some non-limiting examples, rehydrated formulation exhibits a PSD that is substantially identical to, or the same as, that of the carrier oil droplets present in the cannabinoid emulsion as described above when

In other non-limiting examples, the cannabinoid dry formulation may be subjected to a variety of processing steps to form a variety of oral dosage forms for use in cannabis infused products, such as but not limited to tableting processes to form tablets for which at least a fraction of the tablets comprises the cannabinoid dry formulation, coating processes to form a variety of coated particles in which at least a fraction of the coating comprises the cannabinoid dry formulation.

By properly controlling each one of the atomization, evaporation and collection steps above, the present disclosure enables the preparation of a cannabinoid dry formulation produced by spray drying of a cannabinoid emulsion mixed with a solubilized sugar carrier, the cannabinoid dry formulation being suitable for use in cannabis infused products and being re-hydratable, the rehydrated formulation retaining at least some of the characteristics of the cannabinoid emulsion.

In some non-limiting embodiments, the herein described procedures afford a cannabis-infused product which incorporates the cannabinoids in a stable manner. In other words, the cannabis-infused product advantageously remains stable in that there is close to no deterioration of the product appearance within the expected storage shelf-life.

In some embodiments, a beverage cannabis-infused product as provided herein may be stable for at least about 1 month at 4° C. In some embodiments, the cannabis-infused product provided herein may be stable for at least about 2 months at 4° C. In some embodiments, the cannabis-infused product provided herein may be stable for at least about 3 months at 4° C. In some embodiments, the cannabis-infused product provided herein may be stable for at least about 4 months at 4° C. In some embodiments, the cannabis-infused product provided herein may be stable for at least about 5 months at 4° C. In some embodiments, the cannabis-infused product provided herein may be stable for at least about 6 months at 4° C. In some embodiments, the cannabis-infused product provided herein may be stable for at least about 7 months at 4° C. In some embodiments, the cannabis-infused product provided herein may be stable for at least about 8 months at 4° C. In some embodiments, the cannabis-infused product provided herein may be stable for at least about 9 months at 4° C. In some embodiments, the cannabis-infused product provided herein may be stable for at least about 10 months at 4° C. In some embodiments, the cannabis-infused product provided herein may be stable for at least about 11 months at 4° C. In some embodiments, the cannabis-infused product provided herein may be stable for at least about 1 year at 4° C.

In some embodiments, a cannabis-infused product provided herein may be stable for at least about 1 month at room temperature. In some embodiments, a cannabis-infused product provided herein may be stable for at least about 2 months at room temperature. In some embodiments, a cannabis-infused product provided herein may be stable for at least about 3 months at room temperature. In some embodiments, a cannabis-infused product provided herein may be stable for at least about 4 months at room temperature. In some embodiments, a cannabis-infused product provided herein may be stable for at least about 5 months at room temperature. In some embodiments, a cannabis-infused product provided herein may be stable for at least about 6 months at room temperature. In some embodiments, a cannabis-infused product provided herein may be stable for at least about 7 months at room temperature. In some embodiments, a cannabis-infused product provided herein may be stable for at least about 8 months at room temperature. In some embodiments, a cannabis-infused product provided herein may be stable for at least about 9 months at room temperature. In some embodiments, a cannabis-infused product provided herein may be stable for at least about 10 months at room temperature. In some embodiments, a cannabis-infused product provided herein may be stable for at least about 11 months at room temperature. In some embodiments, a cannabis-infused product provided herein may be stable for at least about 1 year at room temperature.

### EXAMPLES

The following examples describe some example modes of making and practicing certain compositions that are described herein. It should be understood that these examples are for illustrative purposes only and are not meant to limit the scope of the compositions and methods described herein.

### Example 1

In this example, microencapsulation compositions containing an emulsion having particle sizes >1000 nm (Formulation 1), 200 nm (Formulation 2) and 40 nm (Formulation 3) were made.

Cannabinoid based emulsions having a particle size of 40 nm and 200 nm are provided below in Tables 1 and 2. Cannabinoid based emulsions having a particle size of >1000 nm were prepared based on the formulae set out in Tables 1 and 2, without the additional sonication step. These example formulations span the range from nano-emulsions to macro-emulsions. The foregoing emulsions were prepared as follows:
The water and oil phase ingredients were solubilized separately using heat and stirring. In particular, the water phase is comprised of water, Tween™ 80, ascorbic acid and EDTA and mixed at 60° C. with a magnetic stir bar for 30 minutes. The oil phase is comprised of Labrafac™ lipophile WL 1349, Tocobiol™, lecithin and THC distillate and mixed at 60° C. with a magnetic stir bar for 30 minutes.
Once the respective water and oil phases have been prepared they were combined while mixing with a high shear homogenizer at 8000-10000 rpm. The oil phase was added slowly to the water phase over 5 minutes and once completely the resultant emulsion was mixed for an additional 15 minutes. The resultant mixture is a macro-emulsion with a particle size >1000 nm.
To generate the 40 nm and 200 nm nano-emulsions, high energy sonication was applied to the macro-emulsions for 10 minutes with 100% amplitude using an LSP-500 Ultrasonic Processor (Sonomechanics, Florida, USA).

Using the same excipient components and tuning the ratio of emulsifiers to achieve the different particle sizes eliminates the experimental uncertainty in permeation data (see in later example) interpretation that would normally be associated if using different emulsifier combinations to achieve the different particle sizes.

Particle size of all nanoemulsions was measured in water solution at 25° C. using dynamic light scattering (DLS). All samples in the present disclosure have been analyzed at a dilution of 1/20 in purified water using a LiteSizer™ (Anton Paar GmbH, Germany).

TABLE 1

| Excipients | Mass (g) | % Blend |
|---|---|---|
| THC Distillate-03 | 18.75 | 2.5 |
| Labrafac lipophile | 20 | 2.67 |
| Ascorbic acid | 4.5 | 0.6 |
| Tocobiol | 3.75 | 0.5 |
| EDTA | 0.1 | 0.01 |
| Lecithin | 15 | 2 |
| Tween 80 | 60 | 8 |
| Water | 627.9 | 83.72 |

TABLE 2

| Excipients | Mass (g) | % Blend |
|---|---|---|
| THC Distillate-03 | 18.75 | 2.5 |
| Labrafac lipophile | 20 | 2.67 |
| Ascorbic acid | 3.75 | 0.5 |
| Tocobiol | 4.5 | 0.6 |
| EDTA | 0.1 | 0.01 |
| Lecithin | 10 | 1.33 |
| Tween80 | 15 | 2 |
| Water | 677.9 | 90.39 |

It was discovered that different particle sizes of the emulsions were achieved by tuning the ratio of the plurality of emulsifiers present in the emulsification system. In particular, they found that a higher concentration of the high HLB value emulsifiers (e.g., Tween™ 80) relative to the low HLB value emulsifiers (e.g., Lecithin, Tocobiol™) generated smaller particle size (e.g., 40 nm) nano-emulsions. Conversely, it was discovered that a higher concentration of the low HLB value emulsifiers relative to the high HLB value emulsifiers resulted in the larger particle size (e.g., 200 nm) nano-emulsions. The results clearly demonstrate that the emulsification approach of the present disclosure allows for tuning the ratio of the emulsifiers to achieve different particle sizes suitable for formulating with a variety of product bases. Additionally, it eliminates the experimental uncertainty that would normally be associated with using different emulsifier combinations to achieve different particle sizes.

### Example 2

In this example, a microencapsulation composition containing THC with a particle size <100 nm was made.

1,000 mg of THC-containing cannabis oil was mixed with 50 mg of poly(ethylene glycol) monooleate with an appropriate amount of ethanol in a container to obtain an oil phase mixture. The oil phase mixture was heated at 50° C. until a liquid oil phase was obtained. In a separate container, 50 mg of sodium oleate were dissolved into 20 mL of deionized water to form an aqueous phase mixture. The oil phase mixture was added to the aqueous phase mixture and the combined mixture was mixed with a high shear mixer to obtain a coarse emulsion. A T25 (IKA, Staufen, Germany) at 8,000 rpm for 5 minutes can be used here. The coarse emulsion was mixed with a microfluidizer to further homogenize the emulsion and obtain the first microencapsulation composition containing THC with a particle size <100 nm. A Nano DeBEE, (Westwood, Mass., USA) at 20,000 psi for 8-12 cycles can be used here.

### Example 3

In this example, a microencapsulation composition containing CBD with a PSD of about 200 nm was made.

5 g of limonene and 25 g of whey protein isolate were mixed with 70 g of water by stirring. The mixture was left for 24 hours to allow complete biopolymer hydration and saturation. After 24 hours, the mixture was homogenized using a sonicator. A Digital Sonifier 450 (Branson Ultrasonic Corporation, USA) at 160 W for 2 minutes can be used here. After homogenization, the emulsion was placed in an ice bath until the emulsion reached room temperature so as to obtain the second microencapsulation composition containing CBD with a PSD of about 200 nm.

### Example 4

In this example, a second microencapsulation composition containing CBD with a PSD of about 200 nm was made.

5 g of CBD-containing cannabis oil extract was mixed with 0.794 g Tween 80, 4.206 g Span 80, and 90 g distilled water in a test tube. The resulting mixture was heated to 70° C. and immediately homogenized to obtain the second microencapsulation composition containing CBD with a PSD of about 200 nm. An Ultra Turrax T 25 device (IKA, Staufen, Germany) at 13,400 rpm for 15 minutes can be used here.

### Example 5

In this example, a second microencapsulation composition containing CBD with a PSD of about 200 nm was made.

0.794 g Tween 80 was dissolved in 90 g distilled water to form an aqueous phase. 4.206 g Span 80 was dissolved in 5 g CBD cannabis oil to form an oil phase. Both the aqueous and oil phases were heated to 70° C. and maintained at this temperature. The aqueous phase was added drop-wise to the oil phase, while stirring the oil phase to obtain the second microencapsulation composition containing CBD with a PSD of about 200 nm. An RZR Heidolph homogenizer (Heidolph Instruments GmbH & Co. KG, Schwabach, Germany) at 1050 rpm over a duration of 30 min can be used here.

### Example 6

In this example, a second microencapsulation composition containing CBD with a PSD of about 200 nm was made.

The same procedure as described in Example 5 was repeated except that 1.262 g Tween 80 was dissolved in 90 g distilled water to form the aqueous phase and 3.738 g Span 80 was dissolved in 5 g CBD cannabis oil extract to form the oil phase.

### Example 7

In this example, a second microencapsulation composition containing CBD with a PSD of about 200 nm was made.

The same procedure as described in Example 5 was repeated except that 1.729 g Tween 80 was dissolved in 90 g distilled water to form the aqueous phase and 3.271 g Span 80 was dissolved in 5 g CBD cannabis oil extract to form the oil phase.

### Example 8

In this example, a second microencapsulation composition containing CBD with a PSD of about 200 nm was made.

The same procedure as described in Example 5 was repeated except that 2.196 g Tween 80 was dissolved in 90 g distilled water to form the aqueous phase and 2.804 g Span 80 was dissolved in 5 g CBD cannabis oil extract to form the oil phase.

### Example 9

In this example, a second microencapsulation composition containing CBD with a PSD of about 200 nm was made.

The same procedure as described in Example 5 was repeated except that 2.664 g Tween 80 was dissolved in 90 g distilled water to form the aqueous phase and 2.336 g Span 80 was dissolved in 5 g CBD cannabis oil extract to form the oil phase.

### Example 10

In this example, a second microencapsulation composition containing CBD with a PSD of about 200 nm was made.

The same procedure as described in Example 5 was repeated except that 2.826 g Tween 80 was dissolved in 90 g distilled water to form the aqueous phase and 2.174 g Span 80 was dissolved in 5 g CBD cannabis oil extract to form the oil phase.

### Example 11

In this example, a second microencapsulation composition containing CBD with a PSD of about 200 nm was made.

The same procedure as described in Example 5 was repeated except that 3.370 g Tween 80 was dissolved in 90 g distilled water to form the aqueous phase and 1.630 g Span 80 was dissolved in 5 g CBD cannabis oil extract to form the oil phase.

### Example 12

In this example, a second microencapsulation composition containing CBD with a PSD of about 200 nm was made (Formulation K).

The same procedure as described in Example 5 was repeated except that 3.913 g Tween 80 was dissolved in 90 g distilled water to form the aqueous phase and 1.087 g Span 80 was dissolved in 5 g CBD cannabis oil extract to form the oil phase.

### Example 13—Mucolytic Agent

In this example, a microencapsulation composition containing THC and a mucolytic agent was made.

Kollipor EL (30% w/w) as surfactant and propylene glycol (47% w/w) as co-solvent were mixed with THC (3% w/w) at 40° C. for 30 minutes using a magnetic stirrer (Hotplate Stirrer Stuart) at the rate of 200 rpm. Captex 355 as oil (20% w/w) was added to this mixture and stirred for a further 30 min at 40° C. at 500 rpm. This mixture was dispersed in 0.1 M phosphate buffered saline solution (pH 6.8) with a volume ratio of 1:100 by stirring at 50 rpm. Papain-palmitate was dispersed in oleic acid at a concentration of 10% (m/v), and subsequently, equal volume of papain-palmitate dispersion and phosphate-buffered mixture were mixed at vortex for 10 min followed by sonication for 6 h at room temperature using Bandelin Sonorex at a frequency of 35 kHz. Droplet-sized particles were immediately observed after dispersing in 0.1 M phosphate buffer solution (pH 6.8) at a volume ratio of 1:100.

Papain-palmitate was prepared according to the following procedure: Papain was dissolved in 0.1 M phosphate buffer (pH 8.0) at a concentration of 3 mg/ml using a thermomixer. Palmitoyl chloride solution in acetone at a concentration of 100 mg/ml was added dropwise into the papain solution at a volume ratio of 1:40. The pH was maintained at 8 by addition of 1 M NaOH. The reaction was conducted for 90 min at room temperature and produced a suspension. Afterwards, the modified papain suspension was dialyzed against water for 24 h followed by lyophilization.

This procedure for incorporating a mucolytic agent can be performed with any of the microencapsulation compositions described in the examples.

### Example 14—Efflux Blocker

In this example, a microencapsulation composition containing a cannabinoid and an efflux blocker was made.

504 mg of polysorbate 20, 504 mg of sorbitan monoleate, 504 mg of polyoxyl 40-hydroxy castor oil, and 504 mg of tricaprin were mixed in a container. In a separate container, 996 mg of ethyl lactate and 254 mg of lecithin were mixed and heated to 40° C. in a scintillation tube until complete dissolution. Both mixtures were mixed together using gentle stirring. The combined mixture was heated to 40° C. until a homogenous pre-concentrate solution was formed. 103 mg of cannabis oil was added to the pre-concentrate solution. The combined mixture was stirred gently, where upon gentle agitation of the cannabinoid in the aqueous phase, the pre-concentrate spontaneously forms drug encapsulated O/W nano-dispersion. 69 mg of an efflux blocker was added to form an advanced pro-nanoparticulates and the mixture was heated to 40° C. until a homogenous solution was formed.

This procedure for incorporating an efflux blocker can be performed with any of the microencapsulation compositions described in the examples.

### Example 15

In this example, various microencapsulation compositions containing THC at 2.5 wt. % were made in accordance with embodiments of the present disclosure and as per the procedure set forth in Example 1.

TABLE 3

| Ingredient | Mass (g) | % Blend |
|---|---|---|
| THC Distillate | 3.75 | 2.50 |
| Coconut Oil | 4 | 2.67 |
| Lecithin sunflower | 3 | 2 |
| Tween 80 | 12 | 8 |
| Water | 127.25 | 84.83 |

| | |
|---|---|
| PSD | 59.4 nm |

TABLE 4

| Ingredients | Mass (g) | % Blend |
|---|---|---|
| THC Distillate | 3.75 | 2.50 |
| Coconut Oil | 4.00 | 2.67 |
| Span 80 | 3.00 | 2.00 |
| Tween 80 | 12.00 | 8.00 |
| Water | 127.25 | 84.83 |

| | |
|---|---|
| PSD | 122.7 nm |

TABLE 5

| Ingredients | Mass (g) | % Blend |
|---|---|---|
| THC Distillate | 3.75 | 2.50 |
| Coconut Oil | 4.00 | 2.67 |
| Brij ™ C2-SO | 1.50 | 1.00 |
| Tween 80 | 11.00 | 7.33 |
| Water (g): | 129.75 | 86.50 |

| | |
|---|---|
| PSD | 87.4 nm |

TABLE 6

| Ingredients | Mass (g) | % Blend |
|---|---|---|
| THC Distillate | 3.75 | 2.50 |
| Coconut Oil | 4.00 | 2.67 |
| Vit E TPGS | 3.00 | 2.00 |
| Tween 80 | 9.00 | 6.00 |
| Lecithin sunflower | 3.00 | 2.00 |
| Water | 127.25 | 84.83 |

| | |
|---|---|
| PSD | 36 nm |

TABLE 7

| Ingredients | Mass (g) | % Blend |
|---|---|---|
| THC Distillate | 3.75 | 1.74 |
| Vit E TPGS | 3.75 | 1.74 |
| Ethanol | 8.00 | 3.71 |
| Tween 20 | 150.00 | 69.61 |
| Water | 50.00 | 23.20 |

| | |
|---|---|
| PSD | 10 nm |

### Example 16—Precursor Composition

In this example, a precursor composition in accordance with an embodiment of the present disclosure was made by gently mixing a microencapsulation composition containing THC with a particle size <100 nm (as described in any one of the previous examples) and a microencapsulation composition containing CBD with a particle size >200 nm (as described in any one of the previous examples).

The microencapsulation compositions were gently mixed to obtain a precursor composition in accordance with an embodiment of the present disclosure.

### Example 17

A THC precursor composition obtained as per the procedure set out in Example 16 was incorporated into a beverage base to obtain a cannabis-infused beverage which was canned into a container (e.g., 355 ml can) so as to include 10 mg THC and 100 mg CBD per container in accordance with an embodiment of the present disclosure. This beverage thus contained a fast onset portion with a cannabinoid profile and a delayed onset portion with an antidote, modulator or attenuator of the cannabinoid profile.

It should be appreciated that the drawings and description herein are intended solely for illustrative purposes, and that the present invention is in no way limited to the particular example embodiments explicitly shown in the drawings and described by way of example herein.

What has been described is merely illustrative of the application of principles of embodiments of the present disclosure. Other arrangements and methods can be implemented by those skilled in the art.

1. A container comprising:
a container body holding a liquid;
a cover releasably coupled to the container body to seal the container body;
a dosage device comprising a cannabinoid-containing substance, packaged with the container, to enable controlled addition of the cannabinoid-containing substance to the liquid to produce a cannabinoid-containing liquid for ingestion,
wherein the dosage device is controllable to release none of the cannabinoid-containing substance, a portion of the cannabinoid-containing substance, or all of the cannabinoid-containing substance into the liquid.

2-3. (canceled)

4. The container of claim 1, wherein the dosage device is packaged inside the container body, wherein the container is pressurized when sealed by the cover, wherein the dosage device is pressure-sensitive and is activated, by opening of the container, to release the cannabinoid-containing substance into the liquid.

5-6. (canceled)

7. The container of claim 1, wherein the dosage device is releasably carried by the cover for release from the cover when the container is to be opened.

8. (canceled)

9. The container of claim 1, wherein the dosage device comprises a compartment in the cover, wherein the cover comprises multiple parts that define the compartment and are movable relative to each other, wherein movement of the multiple parts relative to each other controls addition of the cannabinoid-containing substance to the liquid, wherein the multiple parts are movable between a plurality of positions relative to each other, wherein the plurality of positions are associated with respective amounts of the cannabinoid-containing substance to be released into the liquid.

10-14. (canceled)

15. The container of claim 1, wherein the dosage device comprises a mixing chamber to enable mixing of the cannabinoid-containing substance and a portion of the liquid, and to inhibit mixing of the cannabinoid-containing substance and another portion of the liquid.

16-17. (canceled)

18. The container of claim 1, wherein the dosage device comprises indicia of any one or more of:
an amount of the cannabinoid-containing substance to be released into the liquid; and
an estimated cannabinoid concentration in the cannabinoid-containing liquid.

19. The container of claim 1, wherein the dosage device comprises a further cannabinoid-containing substance, and is configured to enable controlled addition of the further cannabinoid-containing substance to the liquid.

20. (canceled)

21. The container of claim 1, wherein the cover comprises a child-resistant feature to restrict access to contents of the container.

22. The container of claim 1, wherein the dosage device comprises a child-resistant feature to restrict access to the cannabinoid-containing substance.

23. (canceled)

24. A method of use of the container of claim 1 for delivery of a cannabinoid-containing liquid for ingestion, the method comprising:
operating the dosage device to add a controlled amount of the cannabinoid-containing substance to the liquid to produce the cannabinoid-containing liquid;
ingesting the cannabinoid-containing liquid.

25. A cover comprising:
a coupling structure to releasably couple the cover to a liquid container;
a seal to seal the container;
a dosage device comprising a cannabinoid-containing substance to enable controlled addition of the cannabinoid-containing substance to a liquid inside the container to produce a cannabinoid-containing liquid for ingestion,
wherein the dosage device is controllable to release none of the cannabinoid-containing substance, a portion of the cannabinoid-containing substance, or all of the cannabinoid-containing substance into the liquid.

26-27. (canceled)

28. The cover of claim 25, wherein the container is pressurized when sealed by the cover, wherein the dosage device is pressure-sensitive and is activated, by opening of the container, to release the cannabinoid-containing substance into the liquid.

29. The cover of claim 25, wherein the dosage device is releasably carried by the cover for release from the cover when the container is to be opened.

30. The cover of claim 25, wherein the dosage device comprises a compartment in the cover, wherein the cover comprises multiple parts that define the compartment and are movable relative to each other, wherein movement of the multiple parts relative to each other controls addition of the cannabinoid-containing substance to the liquid, wherein the multiple parts are movable between a plurality of positions relative to each other, wherein the plurality of positions are associated with respective amounts of the cannabinoid-containing substance to be released into the liquid.

31-35. (canceled)

36. The cover of claim 25, wherein the dosage device comprises a mixing chamber to enable mixing of the cannabinoid-containing substance and a portion of the liquid, and to inhibit mixing of the cannabinoid-containing substance and another portion of the liquid.

37-38. (canceled)

39. The cover of claim 25, wherein the dosage device comprises indicia of any one or more of:
an amount of the cannabinoid-containing substance to be released into the liquid;
an estimated cannabinoid concentration in the cannabinoid-containing liquid.

40. The cover of claim 25, wherein the dosage device comprises a further cannabinoid-containing substance, and is configured to enable controlled addition of the further cannabinoid-containing substance to the liquid.

41. (canceled)

42. The cover of claim 25, further comprising:
a child-resistant feature to restrict access to contents of the container.

43. The cover of claim 25, wherein the dosage device comprises a child-resistant feature to restrict access to the cannabinoid-containing substance.

44. (canceled)

45. A method comprising:
providing a cannabinoid-containing substance; and
adding the cannabinoid-containing substance to a cover, the cover comprising:
a coupling structure to releasably couple the cover to a liquid container;
a seal to seal the container;
a dosage device to enable controlled addition of the cannabinoid-containing substance to a liquid inside the container to produce a cannabinoid-containing liquid for ingestion,
wherein the dosage device is controllable to release none of the cannabinoid-containing substance, a portion of the cannabinoid-containing substance, or all of the cannabinoid-containing substance into the liquid.

46-47. (canceled)

48. A product package comprising:
a plurality of covers according to claim 25.

49. The product package of claim 48, wherein the plurality of covers comprises covers with dosage devices having one or more of: different cannabinoid-containing substances, different amounts of cannabinoid-containing substances, different estimated effects of cannabinoid-containing substances, and different granularity of control.

50. The product package of claim 48, further comprising:
an indicator of an estimate of effect of either or both of the cannabinoid-containing liquid and the cannabinoid-containing substance.

51. The product package of claim 50, wherein the indicator comprises a respective indicator on each of the covers.

52. The product package of claim 49, wherein the indicator comprises an indicator, on the product package, of any one or more of: the different cannabinoid-containing substances, the different amounts of cannabinoid-containing substances, the different estimated effects of cannabinoid-containing substances, and the different granularity of control.
